(19) United States
(12) Patent Application Publication
(10) Pub. No.: US 2026/0242370 A1
(43) Pub. Date: Aug. 20, 2026

(54) FLOW CHEMISTRY PROCESS FOR PREPARING AZA-BICYCLIC HETEROARYL COMPOUNDS

(71) Applicant: ACTINOGEN MEDICAL LIMITED, Sydney, New South Wales (AU)

(72) Inventors: Jason Douglas WILLIAMS, Graz (AT); Michael PRIESCHL, Graz (AT)

(21) Appl. No.: 19/161,127

(22) PCT Filed: Mar. 1, 2024
(86) PCT No.: PCT/AU2024/050170

§ 371 Date: Aug. 29, 2025

(30) Foreign Application Priority Data
Mar. 2, 2023 (AU) 2023900544

(51) Int. Cl. C07D 451/06

(52) U.S. Cl. CPC C07D 451/06

(57) ABSTRACT

The present disclosure relates to a continuous flow process for synthesis of heterocyclic methanone compounds, and in particular 3-substituted, 3-hydroxyl-(8-aza-bicyclo[3.2.1]oct-8-yl)-[5-(1h-pyrazol-4-yl)-thiophen-3-yl]-methanone compounds, and aza-bicyclo intermediates thereof. In particular, the present disclosure also relates to a continuous flow process that can be utilised toward the synthesis of Xanamem.

### FIELD

The present disclosure generally relates to a continuous flow process for synthesis of heterocyclic methanone compounds, and in particular 3′-substituted, 3-hydroxyl-(8-aza-bicyclo[3.2.1]oct-8-yl)-[5-(lh-pyrazol-4-yl)-thiophen-3-yl]-methanone compounds, and aza-bicyclo intermediates thereof.

### BACKGROUND

Synthetic organic chemists have devised many ways for making organic compounds. However, despite the wide scope and variety of known reactions, most were developed, and are generally still practiced, under batch reaction conditions.

In particular, the synthesis of aza-bicyclic heteroaryl moieties, such as nortropinone moieties, is typically practiced under batch conditions. Such aza-bicyclic heteroaryl moieties are frequent intermediates in the synthesis of numerous pharmaceutical compounds, including Xanamem.

Xanamem, also known as UE2343, is an effective inhibitor of 11β-hydroxysteroid dehydrogenase type 1 (11β-HSD1). Due to its inhibitory action and associated reduction of cortisol levels, Xanamem has been proposed as a treatment of Alzheimer's disease and depression.

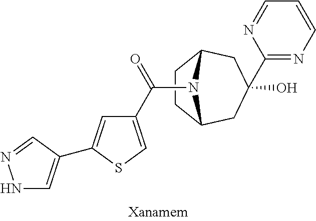 [SMILES: O=C(c1csc(-c2cn[nH]c2)c1)N1[C@@H]2CC[C@H]1C[C@](O)(c1ncccn1)C2]

To date, the reported processes for preparing Xanamem and related analogues comprise coupling of the pyrimidine moiety to an aza-bicyclic heteroaryl moiety, particularly a nortropinone moiety. There is a particular drawback associated with this step, in that the known methods require the use of highly reactive species such as an organolithium reagent. Furthermore, the addition of the organolithium reagent to the reaction mixture results in an exothermic reaction, thereby increasing the temperature of the reaction mixture upon its addition. Due to this, the reaction is typically carefully performed at cryogenic temperatures (e.g. −80° C. or lower) and requires the slow addition of reagents. While such a processing method may be suitable for small-scale synthesis, the reaction does not lend itself to a scale-up process. The required slow addition of reagents leads to scale-dependent reaction behaviour due to the formation of local hot-spots, stoichiometric imbalances, and a build-up of reactive and potentially unstable intermediates. In addition, in preparing large-scale quantities of Xanamem, there is a significant expense and difficulty in maintaining the requisite reaction conditions (e.g., cryogenic reaction temperatures), as well as an amplified safety risk due to thermal runaway.

Accordingly, there remains a need for a safe, efficient, and scalable synthesis of Xanamem and related analogues, where the generation of any undesirable by-products is significantly reduced or avoided, resulting in high yield and purity.

### SUMMARY

It has been surprisingly found that aza-bicyclic heteroaryl compounds may be prepared by a continuous flow process.

In one aspect, there is provided a continuous flow process for preparing an aza-bicyclic compound of Formula 1, or salt thereof:

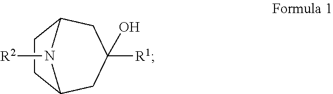

comprising an organolithium reaction of a nortropinone compound of Formula 2:

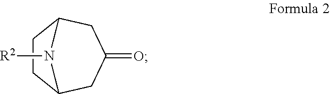

with a halogenated compound of Formula 3:

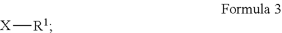

in the presence of an organolithium reagent; wherein R¹ is a monocyclic or bicyclic heteroaryl group each unsubstituted or substituted with one or more substituents selected from the group consisting of halogen, —C₁₋₆alkyl, —O—C₁₋₆alkyl, —C₁₋₆haloalkyl, and —O—C₁₋₆haloalkyl; R² is an amine protecting group; and X is a halogen.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### BRIEF DESCRIPTION OF DRAWINGS

FIG. 1 depicts a calibration curve showing linear response (and gradient) between substance quantity ratio and HPLC area ratio of N-boc-nortropinone against biphenyl (internal standard).

FIG. 2 depicts a calibration showing linear response (and gradient) between substance quantity ratio and HPLC area ratio of 2-iodopyrimidine against biphenyl (internal standard).

FIG. 3 depicts a calibration curve showing linear response (and gradient) between substance quantity ratio and HPLC area ratio of product EU1G against biphenyl (internal standard).

FIG. 4 depicts an example calibration chromatogram, showing all three reaction components and biphenyl (internal standard).

FIG. 5 depicts experimental setup A.

FIG. 6 depicts experimental setup B.

FIG. 7 depicts experimental setup C.

FIG. 8 depicts a chromatogram of the reaction mixture after the flow reaction using MeLi·LiBr. All known substances have been marked. The peak at 3.95 min is likely to be a product of the reaction of MeLi with N-boc-nortropinone. The small side product peak at 3.44 could not be assigned to a structure.

FIG. 9 depicts a chromatogram of the reaction mixture after the flow reaction using HexLi. In the reaction with HexLi, no peak at 3.95 min is observed, but instead a peak at 9.76 min, which most likely corresponds to an addition of HexLi to N-boc-nortropinone.

FIG. 10 depicts flow scheme setup D, showing all flow rates and equivalents utilized for Example 7: Demonstration of Stability in Longer Runs.

FIG. 11 depicts the ¹H-NMR spectrum (DMSO-d₆) of EU1H2·2pTSA from the first flow isolation experiment (Example 9). Purity assessment in triplicate using 1,3,5-trimethoxybenzene as internal standard (95% NMR purity).

FIG. 12 depicts the HPLC trace at 233 nm of EU1H2·2pTSA isolated from the first flow isolation experiment (Example 9; 95% NMR purity). HPLC Method A was used, due to the high polarity of the analytes.

FIG. 13 depicts the ¹H-NMR spectrum (DMSO-d₆) of EU1H2·2pTSA from the second flow isolation experiment (Example 9). Purity assessment in triplicate using 1,3,5-trimethoxybenzene as internal standard (91% NMR purity).

FIG. 14 depicts the HPLC trace at 233 nm of EU1H2.2pTSA isolated from the second flow isolation experiment (Example 9; 91% NMR purity). HPLC Method A was used, due to the high polarity of the analytes.

### DETAILED DESCRIPTION

### General Definitions

Unless specifically defined otherwise, all technical and scientific terms used herein shall be taken to have the same meaning as commonly understood by one of ordinary skill in the art (e.g., chemistry, biochemistry, medicinal chemistry, microbiology and the like).

Unless specifically stated otherwise, or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

As used herein, the term about, unless stated to the contrary, refers to +/−20%, typically +/−10%, typically +/−5%, of the designated value.

The compounds of the present disclosure may contain chiral (asymmetric) centres or the molecule as a whole may be chiral. The individual stereoisomers (enantiomers and diastereoisomers) and mixtures of these are within the scope of the present invention.

As used herein, the term “halogen” means fluorine, chorine, bromine, or iodine.

As used herein, the term “alkyl” encompasses both straight chain (i.e., linear) and branched chain hydrocarbon groups. Examples of alkyl groups include methyl, ethyl, n-propyl, iso-propyl, n-butyl, t-butyl, i-butyl, sec-butyl, pentyl, and hexyl groups. In one example, the alkyl group is of one to six carbon atoms (i.e., C₁₋₆alkyl).

As used herein, the term “carbocyclyl” refers to an aromatic or non-aromatic cyclic group of carbon atoms. A carbocyclyl group may, for example, be monocyclic or polycyclic (i.e. bi-cyclic, tricyclic). A polycyclic carbocyclyl group may contain fused rings. In one example, the carbocyclyl group is of three to ten carbon atoms (i.e. C₃₋₁₀carbocyclyl). Examples of monocyclic non-aromatic carbocyclyl groups include cyclopropyl, cyclobutyl, cyclopentyl, cyclopentenyl, cyclohexyl, cyclohexenyl, cycloheptyl, and cyclooctyl groups. Aromatic carbocyclyl groups include phenyl and napthalenyl.

As used herein, the term “heterocyclyl” refers to an aromatic or non-aromatic cyclic group which is analogous to a carbocyclic group, but in which from one to three of the carbon atoms is/are replaced by one or more heteroatoms independently selected from nitrogen, oxygen, or sulfur. A heterocyclyl group may be, for example, monocyclic or polycyclic (e.g. bicyclic). A polycyclic heterocyclyl may for example contain fused rings. In a bicyclic heterocyclyl group there may be one or more heteroatoms in each ring, or heteroatoms only in one of the rings. A heteroatom may be N, O, or S. Heterocyclyl groups containing a suitable nitrogen atom include the corresponding N-oxides. In one example, the heterocyclyl group is of three to ten atoms (i.e. 3-10-membered heterocyclyl). Examples of monocyclic non-aromatic heterocyclyl groups include aziridinyl, azetidinyl, pyrrolidinyl, imidazolidinyl, py-razolidinyl, piperidinyl, piperazinyl, tetrahydrofuranyl, tetrahydropyranyl, morpholinyl, thi-omorpholinyl and azepanyl. Examples of bicyclic heterocyclyl groups in which one of the rings is non-aromatic include dihydrobenzofuranyl, indanyl, indolinyl, isoindolinyl, tetrahydroisoquinolinyl, tetrahydroquinolyl, and benzoazepanyl. Examples of monocyclic aromatic heterocyclyl groups (also referred to as monocyclic heteroaryl groups) include furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidine. Examples of bicyclic aromatic heterocyclyl groups (also referred to as bicyclic heteroaryl groups) include quinoxalinyl, quinazolinul, pyridopyrazinyl, benzoxazolyl, benzothiophenyl, ben-zimidazolyl, naphthyridinyl, quinolinyl, benzofuranyl, indolyl, benzothiazolyl, oxazolyl[4,5-b]pyridyl, pyridopyrimidinyl, isoquinolinyl, and benzohydroxazole.

As used herein, the term “anion” refers to an ion bearing a negative charge. Similarly, as used herein, the term “cation” refers to an ion bearing a positive charge.

The present disclosure relates to a continuous flow process for preparing an aza-bicyclic compound of Formula 1 and salts thereof. Salts may be formed in the case of embodiments of the compound of Formula 1, which contain a suitable acidic or basic group. Suitable salts of the compound of Formula 1 include those formed with organic or inorganic acids or bases. As used herein, the phrase “pharmaceutically acceptable salt” refers to pharmaceutically acceptable organic or inorganic salts. Exemplary acid addition salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or tri-ethanolamine. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine. It will be understood that the present disclosure encompasses solvated forms, including hydrates, of the compounds of Formula 1 and salts thereof.

Those skilled in the art of organic and/or medicinal chemistry will appreciate that the compounds of Formula 1 and salts thereof may be present in amorphous form, or in a crystalline form. It will be understood that the present disclosure encompasses all forms and polymorphs of the compounds of Formula 1 and salts thereof.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be clearly understood that, although a number of prior art publications are referred to herein, this reference does not constitute an admission that any of these documents forms part of the common general knowledge in the art, in Australia or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will prevail. In addition, the materials, methods, and examples are illustrative only and not intended to be limiting.

### Continuous Flow Process

The subject matter of the present disclosure is predicated in part on the surprising discovery of an efficient and scalable process for preparing an aza-bicyclic compound of Formula 1 via a continuous flow process.

Accordingly, there is provided a continuous flow process for preparing an aza-bicyclic compound of Formula 1, or salt thereof:

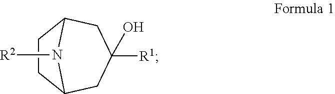

comprising an organolithium reaction of a nortropinone compound of Formula 2:

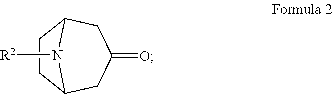

with a halogenated compound of Formula 3:

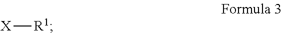

in the presence of an organolithium reagent; wherein R¹ is a monocyclic or bicyclic heteroaryl group each unsubstituted or substituted with one or more substituents selected from the group consisting of halogen, —C₁₋₆alkyl, —O—C₁₋₆alkyl, —C₁₋₆haloalkyl, and —O—C₁₋₆haloalkyl; R² is an amine protecting group; and X is a halogen.

### Continuous Flow Reactor

Utilising a continuous flow reactor, commonly referred to as simply a flow reactor, for the synthesis of small molecules offers an alternative to traditional batch production chemical reactions. However, while continuous flow reactors are finding increasing application in chemical and biological processes, their application in pharmaceutical manufacturing is rare.

A continuous flow reactor allows for materials (e.g., chemicals) to be introduced into and carried through the system as fluid in a flowing stream in one or more conduits within the reactor. As used herein, a “conduit” may refer to any pipe, tube, channel, channelled plate, or any other vessel of suitable shape for conveying fluids in a continuous flow process. A system of pumps propel the flowing streams through the reactor, ultimately combining one or more flowing streams where the fluids come into contact and undergo a chemical reaction to produce a desired chemical(s) (e.g., small molecule(s)). Ultimately, the reactor can be configured to result in the output of a single flowing stream comprising the desired chemical(s).

Undertaking chemical syntheses in a continuous flow reactor may provide one or more advantages over conventional batch or semi-batch syntheses. In particular, the use of a flow reactor allows for reaction variables—such as concentration, flow rate, residence time, temperature, etc.—to be closely monitored and adjusted as required so as to optimise conversion of starting materials and reagents to the desired product. In one example, the continuous flow reaction described herein allows for the adjustment of one or more rection variables selected from the group consisting of molar ratio of reagents, concentration of reagents, flow rate, residence time, temperature, solvent, and output rate.

As used herein, the term “feed concentration” refers to the concentration of a reagent prior to its introduction into a conduit within the continuous flow reactor. Feed concentration is measured in mol/L.

As used herein, the term “flow rate” refers to volumetric flow rate, which is defined as a measure of the volume of liquid which passes per unit time. That is to say, it refers to the speed at which the flowing stream moves through a conduit within the reactor. Flow rate is measured in L/min.

As used herein, the term “residence time” refers to the period of time that a fluid parcel of the flow stream (and consequently the reagents contained therein) resides in a particular segment of the continuous flow reactor. As used herein, “fluid parcel” refers to a very small amount of fluid, identifiable throughout its dynamic history while moving with the flow stream. The residence time is therefore a function of reactor volume and flow rate. Indeed, residence time may be calculated from the ratio of the segment volume and the rate of flow through that segment. It will be understood that particular segments and conduits of a reactor may give rise to different residence times, by virtue of, for example, the volume of the segment, and/or the rate of flow within that segment or conduit. It will be understood that one means by which to achieve a longer residence time, will be to decrease flow rate. Similarly, it will be understood that one means by which to achieve a shorter residence time, will be to increase flow rate. Residence time is typically measured in seconds.

While such continuous flow reactors have previously been best suited to small-scale chemical reactions, the continuous output of a single flowing stream comprising the desired chemical(s) enables application on larger scales, in a scale-independent manner. As such, the utilisation of continuous flow reactors in the synthesis of chemicals, particularly small molecules, is an emerging area of interest in both research and industrial/commercial laboratories.

### Compounds of Formula 1

Aza-bicyclic compounds of Formula 1, or salts thereof, are prepared in a continuous flow process by an organolithium reaction of a nortropinone compound of Formula 2 with a halogenated compound of Formula 3, in the presence of an organolithium reagent.

The continuous flow process described herein prepares an aza-bicyclic compound of Formula 1, or salt thereof:

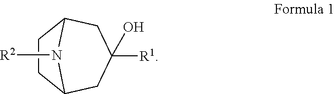

In the aza-bicyclic compound of Formula 1, or salt thereof, R¹ is a monocyclic or bicyclic heteroaryl group each unsubstituted or substituted with one or more substituents selected from the group consisting of halogen, —C₁₋₆alkyl, —O—C₁₋₆alkyl, C₁₋₆haloalkyl, and —O—C₁₋₆haloalkyl. In one example, R¹ is a monocyclic heteroaryl group. In one example, R¹ is a bicyclic heteroaryl group. The cyclic or bicyclic heteroaryl group may be unsubstituted or substituted. In one example, R¹ is an unsubstituted cyclic heteroaryl group. In one example, R¹ is an unsubstituted bicyclic heteroaryl group. In one example, R¹ is a substituted monocyclic heteroaryl group. In one example, R¹ is a substituted bicyclic heteroaryl group.

If substituted, the monocyclic or bicyclic heteroaryl group may be substituted with one or more substituents selected from the group consisting of halogen, —C₁₋₆alkyl, —O—C₁₋₆alkyl, C₁₋₆haloalkyl, and —O—C₁₋₆haloalkyl. In some embodiments, R¹ is a monocyclic heteroaryl group substituted with one or more substituents selected from the group consisting of halogen, —C₁₋₆alkyl, —O—C₁₋₆alkyl, C₁₋₆haloalkyl, and —O—C₁₋₆haloalkyl. In some embodiments, R¹ is a bicyclic heteroaryl group substituted with one or more substituents selected from the group consisting of halogen, —C₁₋₆alkyl, —O—C₁₋₆alkyl, C₁₋₆haloalkyl, and —O—C₁₋₆haloalkyl. In one example, R¹ is a monocyclic heteroaryl group substituted with halogen. In one example, R¹ is a monocyclic heteroaryl group substituted with —C₁₋₆-alkyl. In one example, R¹ is a monocyclic heteroaryl group substituted with —O—C₁₋₆alkyl. In one example, R¹ is a monocyclic heteroaryl group substituted with C₁₋₆haloalkyl. In one example, R¹ is a monocyclic heteroaryl group substituted with —O—C₁₋₆haloalkyl. In one example, R¹ is a bicyclic heteroaryl group substituted with halogen. In one example, R¹ is a bicyclic heteroaryl group substituted with —C₁₋₆-alkyl. In one example, R¹ is a bicyclic heteroaryl group substituted with —O—C₁₋₆alkyl. In one example, R¹ is a bicyclic heteroaryl group substituted with C₁₋₆haloalkyl. In one example, R¹ is a bicyclic heteroaryl group substituted with —O—C₁₋₆haloalkyl. In one example, R¹ is an unsubstituted pyrimidine group.

In the aza-bicyclic compound of Formula 1, or salt thereof, R² is an amine protecting group. The term “amine protecting group” specifically refers to a protecting group that chemically modifies an amine functional group to obtain chemoselectivity in a subsequent chemical reaction. Examples of amine protecting groups include, but are not limited to, carbamate, amide, benzyl, benzylidene, tosyl, and trityl protecting groups. In some embodiments, R² is an amino protecting group selected from the group consisting of a carbamate, amide, benzyl, benzylidene, tosyl, and trityl protecting group. Examples of carbamate protecting groups include, but are not limited to, methyl and ethyl groups, 9-fluoroenylmethyl, 9-fluoroenylmethyloxycarbonyl (Fmoc), tert-butyloxycarbonyl (Boc), benzyl carbamate (Cbz), and p-methoxybenzyl carbonyl (MeOZ) groups. In some embodiments, R² is a tert-butyloxycarbonyl (Boc) protecting group. Examples of amide protecting groups include, but are not limited to, acetyl (Ac), benzamide, trifluoroacetamide, trichloroacetamide, phenylacetamide, picolinamide, and phthalimide groups. Further examples of amino protecting groups include, but are not limited to, benzoyl, benzyl, benzylidene, p-methoxybenzyl (PMB), 3,4-dimethoxybenzyl (DMPM), p-methoxyphenyl (PMP), tosyl (Ts), trichloroethyl chloroformate (Troc), toluene sulphonyl, trityl, and triphenylmethyl groups. Accordingly, in some embodiments, R² is an amine protected group selected from the group consisting of methyl and ethyl groups, 9-fluoroenylmethyl, 9-fluoroenylmethyloxycarbonyl (Fmoc), tert-butyloxycarbonyl (Boc), benzyl carbamate (Cbz), and p-methoxybenzyl carbonyl (MeOZ) groups. In some embodiments, R² is a tert-butyloxycarbonyl (Boc) protecting group. Examples of amide protecting groups include, but are not limited to, acetyl (Ac), benzamide, trifluoroacetamide, trichloroacetamide, phenylacetamide, picolinamide, and phthalimide groups. Further examples of amino protecting groups include, but are not limited to, benzoyl, benzyl, benzylidene, p-methoxybenzyl (PMB), 3,4-dimethoxybenzyl (DMPM), p-methoxyphenyl (PMP), tosyl (Ts), trichloroethyl chloroformate (Troc), toluene sulphonyl, trityl, and triphenylmethyl groups. In one example, R² is an amine protecting group that is tert-butyloxycarbonyl (boc). In one example, R² is an amine protecting group that is 9-fluorenylmethyl carbamate FMOC. In one example, R² is an amine protecting group that is benzyl carbamate (CBZ). In one example, R² is an amine protecting group that is acetamide. In one example, R² is an amine protecting group that is trifluoroacetamide. In one example, R² is an amine protecting group that is phthalimide. In one example, R² is an amine protecting group that is benzyl. In one example, R² is an amine protecting group that is benzylidene. In one example, R² is an amine protecting group that is tosyl (e.g. toluene sulphonyl). In one example, R² is an amine protecting group that is trityl (e.g. triphenylmethyl).

### Compounds of Formula 2

The continuous flow process described herein prepares an aza-bicyclic compound of Formula 1, or salt thereof, an organolithium reaction of a notropinone compound of Formula 2:

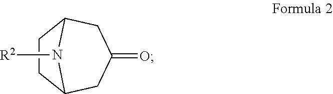

with a halogenated compound of Formula 3 in the presence of an organolithium reagent is undertaken.

In the nortropinone compound of Formula 2, R² is an amine protecting group as described herein. Accordingly, in one example, R² is an amine protecting group that is tert-butyloxycarbonyl (boc). In one example, R² is an amine protecting group that is 9-fluorenylmethyl carbamate (FMOC). In one example, R² is an amine protecting group that is benzyl carbamate (CBZ). In one example, R² is an amine protecting group that is acetamide. In one example, R² is an amine protecting group that is trifluoroacetamide. In one example, R² is an amine protecting group that is phthalimide. In one example, R² is an amine protecting group that is benzyl. In one example, R² is an amine protecting group that is benzylidene. In one example, R² is an amine protecting group that is tosyl (e.g. toluene sulphonyl). In one example, R² is an amine protecting group that is trityl (e.g. triphenylmethyl).

As will be understood by the person skilled in the art, the R² group present in the nortropinone compound of Formula 2 is maintained throughout the organolithium reaction with a halogenated compound to be incorporated as the R² group of the aza-bicyclic compound of Formula 1. For example, when R² of Formula 2 is a tert-butyloxycarbonyl (boc) amine protecting group, R² of the prepared aza-bicyclic compound of Formula 1 is also a tert-butyloxycarbonyl (boc) amine protecting group.

The compound of Formula 2 is provided in any amount suitable for undertaking the continuous flow reaction as described herein. In some embodiments, the amount of the compound of Formula 2, relative to that of the compound of Formula 3, is provided in a Molar ratio of at least about 0.5, 0.6, 0.7, 0.8, 0.9, 1.0, 1.1, 1.15, 1.2, 1.25, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0. In some embodiments, the amount of the compound of Formula 2, relative to that of the compound of Formula 3, is provided in a Molar ratio of less than about 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.25, 1.2, 1.15, 1.1, or 1.0. In some embodiments, the amount of the compound of Formula 2, relative to that of the compound of Formula 3, is in a Molar ratio range provided by any two of the above upper and/or lower amounts, for example, wherein the Molar ratio range is between about 1.0 to 2.0, 1.1 to 1.7, 1.1 to 1.5, 1.15 to 1.25, or 1.2 to 1.3. In one example, the amount of the compound of Formula 2, relative to that of the compound of Formula 3, is in a Molar ratio range of 1.5 to 1.6. In one example, the amount of the compound of Formula 2, relative to that of the compound of Formula 3, is in a Molar ratio range of 1.2 to 1.3. In one example, the amount of the compound of Formula 2, relative to that of the compound of Formula 3, is in a Molar ratio range of 1.15 to 1.25. In one example, the amount of the compound of Formula 2, relative to that of the compound of Formula 3, is in a Molar ratio of about 1.15. In one example, the amount of the compound of Formula 2, relative to that of the compound of Formula 3, is in a Molar ratio of about 1.25.

The compound of Formula 2 concentration may be configured to be at any concentration suitable for undertaking the continuous flow reaction as described herein. In some embodiments, the compound of Formula 2 is provided in a concentration (in mol/L) of at least about 0.1, 0.2, 0.3, 0.4, 0.5, 0.6, 0.7, 0.8, 0.9, 1.0, 1.1, 1.2, 1.3, 1.4, or 1.5 mol/L. In some embodiments, the compound of Formula 2 is provided in a concentration (in mol/L) of less than about 1.6, 1.5, 1.4, 1.3, 1.2, 1.1, 1.0, 0.9, 0.8, 0.7, 0.6, 0.5, 0.4, 0.3, 0.2, or 0.1 mol/L. In some embodiments, the compound of Formula 2 is provided in a concentration (in mol/L) range provided by any two of the previously described upper and/or lower amounts, for example, in a concentration (in mol/L) between about 0.1 to 1.5, 0.2 to 1.2, 0.2 to 0.7, or 0.3 to 0.5 mol/L.

The continuous flow stream comprising the compound of Formula 2 may be configured at any flow rate suitable for undertaking the continuous flow reaction as described herein. In some embodiments, the continuous flow stream comprising the compound of Formula 2 has a flow rate (mL/min) of at least about 0.5, 1.0, 1.5, 2.0, 2.5, 3.0, 3.5, 4.0, 4.5, or 5.0 mL/min. In some embodiments, the continuous flow stream comprising the compound of Formula 2 has a flow rate (mL/min) of less than about 5.0, 4.5, 4.0, 3.5, 3.0, 2.5, 2.0, 1.5, 1.0, or 0.5 mL/min. In some embodiments, the continuous flow stream comprising the compound of Formula 2 has a flow rate in a range provided by any two of the previously described upper and/or lower amounts, for example, a flow rate (mL/min) between about 0.1 and 5, 0.5 and 4.5, 1.0 and 4.0, 1.5 and 3.5, or 2.0 and 3.0 mL/min.

### Compounds of Formula 3

The continuous flow process described herein prepares an aza-bicyclic compound of Formula 1, or salt thereof, from an organolithium reaction of a notropinone compound of Formula 2 with a halogenated compound of Formula 3:

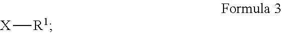

in the presence of an organolithium reagent.

In the halogenated compound of Formula 3, R¹ is a monocyclic or bicyclic heteroaryl group each unsubstituted or substituted with one or more substituents selected from the group consisting of halogen, —OH, —C₁₋₆alkyl, —O—C₁₋₆alkyl, C₁₋₆haloalkyl, and —O—C₁₋₆haloalkyl, as described herein.

If substituted, the monocyclic or bicyclic heteroaryl group may be substituted with one or more substituents selected from the group consisting of halogen, —OH, —C₁₋₆alkyl, —O—C₁₋₆alkyl, C₁₋₆haloalkyl, and —O—C₁₋₆haloalkyl. In some embodiments, R¹ is a monocyclic heteroaryl group substituted with one or more substituents selected from the group consisting of halogen, —OH, —C₁₋₆alkyl, —O—C₁₋₆alkyl, C₁₋₆haloalkyl, and —O—C₁₋₆haloalkyl. In some embodiments, R¹ is a bicyclic heteroaryl group substituted with one or more substituents selected from the group consisting of halogen, —OH, —C₁₋₆alkyl, —O—C₁₋₆alkyl, C₁₋₆haloalkyl, and —O—C₁₋₆haloalkyl. In one example, R¹ is a monocyclic heteroaryl group substituted with halogen. In one example, R¹ is a monocyclic heteroaryl group substituted with —OH. In one example, R¹ is a monocyclic heteroaryl group substituted with —C₁₋₆-alkyl. In one example, R¹ is a monocyclic heteroaryl group substituted with —O—C₁₋₆alkyl. In one example, R¹ is a monocyclic heteroaryl group substituted with C₁₋₆haloalkyl. In one example, R¹ is a monocyclic heteroaryl group substituted with —O—C₁₋₆haloalkyl. In one example, R¹ is a bicyclic heteroaryl group substituted with halogen. In one example, R¹ is a bicyclic heteroaryl group substituted with —OH. In one example, R¹ is a bicyclic heteroaryl group substituted with —C₁₋₆-alkyl. In one example, R¹ is a bicyclic heteroaryl group substituted with —O—C₁₋₆alkyl. In one example, R¹ is a bicyclic heteroaryl group substituted with C₁₋₆haloalkyl. In one example, R¹ is a bicyclic heteroaryl group substituted with —O—C₁₋₆haloalkyl. In one example, R¹ is an unsubstituted pyrimidine group.

The compound of Formula 3 feed concentration may be configured to be at any concentration suitable for undertaking the continuous flow reaction as described herein. In some embodiments, the compound of Formula 3 is provided in a concentration (in mol/L) of at least about 0.1, 0.2, 0.3, 0.4, 0.5, 0.6, 0.7, 0.8, 0.9, 1.0, 1.1, 1.2, 1.3, 1.4, or 1.5 mol/L. In some embodiments, the compound of Formula 3 is provided in a concentration (in mol/L) of less than about 1.6, 1.5, 1.4, 1.3, 1.2, 1.1, 1.0, 0.9, 0.8, 0.7, 0.6, 0.5, 0.4, 0.3, 0.2, or 0.1 mol/L. In some embodiments, the compound of Formula 3 is provided in a concentration (in mol/L) range provided by any two of the previously described upper and/or lower amounts, for example, a concentration (in mol/L) between about 0.1 to 1.5, 0.2 to 1.2, 0.2 to 0.7, or 0.3 to 0.5 mol/L.

The continuous flow stream comprising the compound of Formula 3 may be configured at any flow rate suitable for undertaking the continuous flow reaction as described herein. In some embodiments, the continuous flow stream comprising the compound of Formula 3 has a flow rate (mL/min) of at least about 0.5, 1.0, 1.5, 2.0, 2.5, 3.0, 3.5, 4.0, 4.5, or 5.0 mL/min. In some embodiments, the continuous flow stream comprising the compound of Formula 3 has a flow rate (mL/min) of less than about 5.0, 4.5, 4.0, 3.5, 3.0, 2.5, 2.0, 1.5, 1.0, or 0.5 mL/min. In some embodiments, the continuous flow stream comprising the compound of Formula 3 has a flow rate in a range provided by any two of the previously described upper and/or lower amounts, for example, a flow rate (mL/min) between about 0.1 and 5, 0.5 and 4.5, 1.0 and 4.0, 1.5 and 3.5, or 2.0 and 3.0 mL/min.

In one example, the halogenated compound of Formula 3 is selected from the group consisting of:

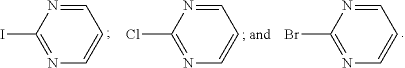

As will be understood by the person skilled in the art, the R¹ group present in the halogenated compound of Formula 3 is maintained throughout the organolithium reaction to be incorporated as the R¹ group of the aza-bicyclic compound of Formula 1. For example, when R¹ of Formula 3 is an unsubstituted pyrimidine group, R¹ of the prepared aza-bicyclic compound of Formula 1 is also an unsubstituted pyrimidine group.

Accordingly, in one example, the halogenated compound of Formula 3 is a compound of Formula 3a:

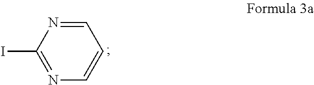

and the aza-bicyclic compound of Formula 1 is a compound of Formula 1a:

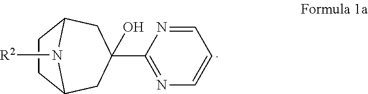

In the halogenated compound of Formula 3, X is a halogen. In some embodiments, X is selected from the group consisting of chlorine, bromine, and iodine. In one example, X is chlorine. In one example, X is bromine. In one example, X is iodine.

In one example, there is provided a continuous flow process for preparing an aza-bicyclic compound of Formula 1a, or salt thereof:

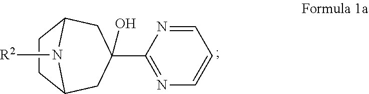

comprising an organolithium reaction of a nortropinone compound of Formula 2:

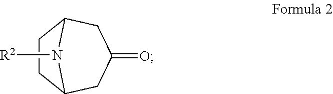

with a halogenated compound of Formula a:

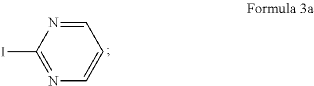

in the presence of an organolithium reagent; wherein R² is an amine protecting group as described herein.

In one example, there is provided a continuous flow process for preparing an

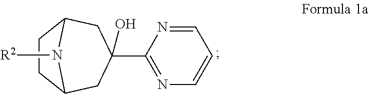

comprising an organolithium reaction of a nortropinone compound of Formula 2:

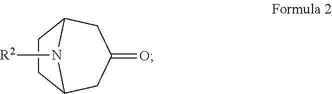

with a halogenated compound of Formula 3a:

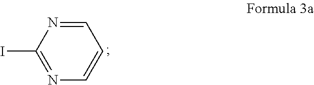

in the presence of an organolithium reagent; wherein R² is a tert-butyloxycarbonyl (boc) amine protecting group.

### Organolithium Reagent

The organolithium reagent utilised in the continuous flow process may be any organolithium reagent suitable for such an organolithium reaction as would be understood by the person skilled in the art. For example, the organolithium reagent may be any compound containing a carbon-lithium bond. In one example, the organolithium reagent is a compound containing a carbon-lithium bond.

Examples of suitable organolithium reagents include, but are not limited to, alkyllithium, alkenyllithium, alkynyllithium, aralkyllithium, aryllithium, heteroaryllithium, alkyllithium magnesium complexes, and any lithium halide complexes thereof. Accordingly, in some embodiments, the organolithium reagent is selected from the group consisting of alkyllithium, alkenyllithium, alkynyllithium, aralkyllithium, aryllithium, heteroaryllithium, alkyllithium magnesium complexes, and any lithium halide complexes thereof.

More specifically, in some embodiments, the organolithium reagent may be selected from the group consisting of methyllithium, ethyllithium, propyllithium, butyllithium, pentyllithium, hexyllithium, methoxymethyllithium, ethoxymethyllithium, vinyllithium, allyllithium, propenyllithium, butenyllithium, ethynyllithium, butynyllithium, pentynyllithium, hexynyllithium, benzyllithium, phenylethyllithium, phenyllithium, naphthyllithium, 2-thienyllithium, 4-pyridyllithium, 2-quinolyllithium, tri(n-butyl)magnesiumlithium and trimethylmagnesiumlithium, (trimethylsilyl)methyllithium, lithium(trimethylsilyl) acetylide, lithium acetylide, lithium phenylacetylide, cyclopentadienyllithium, lithium pentamethylcyclopentadienide, lithium methide, and any lithium halide complexes thereof. In some embodiments, the organolithium reagent is selected from the group consisting of methyllithium or any lithium halide complexes thereof. In one example, the organolithium reagent is methyllithium lithium bromide complex.

As would be appreciated by the person skilled in the art, the organolithium reagent is typically provided in a suitable solvent. In one example, the organolithium reagent is provide in an organic solvent. In one example, the organolithium reagent is provided in an organic solvent selected from the group consisting of 2-methyltetrahydrofuran, diethoxymethane, diethyl ether, tetrahydrofuran, and any combination thereof. In one example, the organolithium reagent is provided in 2-methyltetrahydrofuran solvent. In one example, the organolithium reagent is provided in diethoxymethane solvent. In one example, the organolithium reagent is provided in diethyl ether/tetrahydrofuran solvent. In one example, the organolithium reagent is methyllithium lithium bromide complex, and is provided in 2-methyltetrahydrofuran solvent. In one example, the organolithium reagent is methyllithium lithium bromide complex, and is provided in diethoxymethane solvent. In one example, the organolithium reagent is methyllithium lithium bromide complex, and is provided in diethyl ether/tetrahydrofuran solvent.

The organolithium reagent may be provided in the suitable solvent in a defined amount (mol/L). In some embodiments, the organolithium reagent is provided in an organic solvent in an amount (mol/L) of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0. In some embodiments, the organolithium reagent is provided in an organic solvent in an amount (mol/L) of less than about 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1. In some embodiments, the organolithium reagent is provided in an organic solvent in an amount (mol/L) provided by any two of the previously described upper and/or lower amounts, for example, wherein the amount (mol/L) is between about 0.2 to 1.8 and 0.5 to 1.5. In one example, the organolithium reagent is provided in an organic solvent in an amount (mol/L) of about 1.5. In one example, the organolithium reagent is provided in an organic solvent in an amount (mol/L) of about 0.5. In one example, the organolithium reagent is methyllithium lithium bromide complex, and is provided in diethyl ether/tetrahydrofuran solvent in an amount (mol/L) of about 0.5. In one example, the organolithium reagent is methyllithium lithium bromide complex, and is provided in 2-methyltetrahydrofuran solvent in an amount (mol/L) of about 1.5.

The organolithium reagent is provided in any amount suitable for undertaking the continuous flow reaction as described herein. In some embodiments, the amount of the organolithium reagent relative to the compound of Formula 3 is provided in a Molar ratio of at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0. In some embodiments, the amount of the organolithium reagent relative to compound of Formula 3 is provided in a Molar ratio of less than about 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, or 1.1. In some embodiments, the amount of the organolithium reagent relative to compound of Formula 3 is provided in a Molar ratio range provided by any two of the previously described upper and/or lower amounts, for example, wherein the Molar ratio range is between about 1.0 to 3.0, 1.1 to 1.8, 1.2 to 1.6, or 1.3 to 1.4. In one example, the amount of the organolithium reagent relative to compound of Formula 3 is provided in a Molar ratio range of 1 to 3.

The organolithium reagent feed concentration may be configured to be at any concentration suitable for undertaking the continuous flow reaction as described herein. In some embodiments, the organolithium reagent is provided in a concentration (mol/L) of at least about 0.1, 0.2, 0.3, 0.4, 0.5, 0.6, 0.7, 0.8, 0.9, 1.0, 1.1, 1.2, 1.3, 1.4, or 1.5 mol/L. In some embodiments, the organolithium reagent is provided in a concentration (mol/L) of less than about 1.6, 1.5, 1.4, 1.3, 1.2, 1.1, 1.0, 0.9, 0.8, 0.7, 0.6, 0.5, 0.4, 0.3, 0.2, or 0.1 mol/L. In some embodiments, the organolithium reagent is provided in a concentration range provided by any two of the previously described upper and/or lower amounts, for example, between about 0.1 to 1.5, 0.2 to 1.2, or 0.3 to 0.9 mol/L.

The continuous flow stream comprising the organolithium reagent may be configured at any flow rate suitable for undertaking the continuous flow reaction as described herein. In some embodiments, the continuous flow stream comprising the organolithium reagent has a flow rate (mL/min) of at least about 0.5, 1.0, 1.5, 2.0, 2.5, 3.0, 3.5, 4.0, 4.5, or 5.0 mL/min. In some embodiments, the continuous flow stream comprising the organolithium reagent has a flow rate (mL/min) of less than about 5.0, 4.5, 4.0, 3.5, 3.0, 2.5, 2.0, 1.5, 1.0, or 0.5 mL/min. In some embodiments, the continuous flow stream comprising the organolithium reagent has a flow rate in a range provided by any two of the previously described upper and/or lower amounts, for example, a flow rate (mL/min) of between about 0.1 and 5, 0.5 and 4.5, 1.0 and 4.0, or 2.0 and 3.0 mL/min.

In some embodiments, two or more organolithium reagents are employed in the organolithium reaction. The two or more organolithium reagents may be provided in a single continuous flow stream, or otherwise two or more separate continuous flow streams. In one example, one organolithium reagent is employed, and in a single continuous flow stream. In one example, two organolithium reagents are employed, and in one continuous flow stream (i.e., the two organolithium reagents are combined in a single continuous flow stream). In one example, two organolithium reagents are employed, in two continuous flow streams, with each organolithium reagent provided in a separate continuous flow stream. In one example, methyllithium lithium bromide complex is provided in two or more separate streams. In one example, methyllithium lithium bromide in diethyl ether/tetrahydrofuran is provided in a first stream, and methyllithium lithium bromide in 2-methyltetrahydrofuran is provided in a second stream.

### Quenching

It will be appreciated that, following the organolithium reaction, the resultant solution comprising the aza-bicyclic compound of Formula 1, or salt thereof, may require quenching. As used herein, the term “quenching” refers to the deactivation of any unreacted reagents in a reaction mixture. Accordingly, in some embodiments, the continuous flow process further comprises combining the aza-bicyclic compound of Formula 1, or salt thereof, with a quenching solution. That is, in one example, the resultant solution comprising the aza-bicyclic compound of Formula 1, or salt thereof, is quenched.

The quenching solution may be any suitable solution as would be understood by the person skilled in the art. In some embodiments, the quenching solution is selected from the group consisting of: a polar protic solvent, a non-polar protic solvent, an aqueous solvent, an alcohol, and an acid, or a combination thereof. In one example, the quenching solution is a protic solvent. In one example, the quenching solution comprises a polar protic solvent. In one example, the quenching solution comprises a non-polar protic solvent. In one example, the quenching solution comprises an aqueous solvent. In one example, the quenching solution comprises an alcohol. In one example, the quenching solution comprises isopropanol. In one example, the quenching solution comprises methanol. In one example, the quenching solution comprises benzyl alcohol. In one example, the quenching solution comprises cyclohexanol. In one example, the quenching solution comprises acetone. In one example, the quenching solution comprises an acid. In one example, the quenching solution comprises citric acid. In one example, the quenching solution comprises about 0.25 M citric acid. In one example, the quenching solution comprises phosphoric acid. In one example, the quenching solution comprises sulfuric acid. In one example, the quenching solution comprises ammonium chloride. In one example, the quenching solution comprises benzoic acid. In one example, the quenching solution comprises p-toluenesulfonic acid (pTSA). In one example, the quenching solution comprises acetic acid. In one example, the quenching solution comprises trifluoroacetic acid.

The quenching of the solution comprising the aza-bicyclic compound of Formula 1, or salt thereof, may be undertaken externally to the continuous flow reactor. That is, the output flow stream being a solution comprising the aza-bicyclic compound of Formula 1, or salt thereof, may be collected from the continuous flow reactor, and then separately combined with the quenching solution (i.e., “quenched”). That is, in one example, the quenching of the solution comprising the aza-bicyclic compound of Formula 1 is undertaken externally to the continuous flow reactor.

Alternatively, the quenching of the solution comprising the aza-bicyclic compound of Formula 1 may be undertaken in the continuous flow reactor. Accordingly, in some embodiments, the process comprises combining a continuous flow stream comprising the aza-bicyclic compond of Formula 1, or salt thereof, with a continuous flow stream comprising the quenching solution, to obtain a continuous flow stream comprising the aza-bicyclic compound of Formula 1.

The quenching solution may be provided at any concentration effective to quench the reaction, as would be understood by the person skilled in the art. In some embodiments, the concentration (mol/L) of the quenching solution may be at least about 0.01, 0.05, 0.1, 0.2, 0.3, 0.4, 0.5, 0.6, 0.7, 0.8, 0.9, 1.0, 1.2, 1.4, 1.6, 1.8, 2.0, 3, 4, 5, 6, 7, 8, 9, or 10 mol/L. In some embodiments, the concentration (mol/L) of the quenching solution may be less than about 10, 9, 8, 7, 6, 5, 4, 3, 2, 1, 1.8, 1.6, 1.4, 1.2, 1. 0.9, 0.8, 0.7, 0.6, or 0.5 mol/L. In some embodiments, the concentration (mol/L) of the quenching solution may be in a range provided by any two of the previously described upper and/or lower amounts, for example, between about 0.1 and 10, 0.5 and 8, or 2 and 7 mol/L.

In some embodiments the quenching solution may be anhydrous. For example, the amount of water in the solution may be less than about (in ppm) 500, 400, 300, 200 100, 75, 50, 25, 10, 5, or 1 ppm.

In some embodiments, where the quenching solution is introduced as continuous flow stream, the continuous flow stream has a flow rate (mL/min) of at least about 0.1, 0.5, 1, 2, 3, 4, 5, 6, 7, 8, 9, 10, 20, 30, 40, 50, 100, 200, 300, 400, 500, 1000, 2000, 3000, 4000, 5000, or 10000 mL/min. In some embodiments, the quenching solution is introduced as a continuous flow stream having a flow rate (mL/min) of less than about 10000, 5000, 4000, 3000, 2000, 1000, 500, 400, 300, 200, 100, 50, 40, 30, 20, 10, 9, 8, 7, 6, 5, 4, 3, 2, 1, 0.5, or 0.1 mL/min. In some embodiments, the quenching solution is introduced as continuous flow stream having a flow rate (mL/min) in a range provided by any two of the previously described upper and/or lower amounts, for example, between about 0.1 and 10, 0.5 and 8, or 2 and 7 L/mmin.

It is necessary for a sufficient time to have elapsed prior to the quenching of the solution comprising the aza-bicyclic compound of Formula 1, or salt thereof. That is, it is necessary to provide a sufficient time for the organolithium reaction to occur (and preferably at optimal conversion). As such, it may be necessary, following addition of the organolithium reagent, for the resultant solution to reside for a certain period of time within the reactor so as to allow the organolithium reaction to occur (and preferably at optimal conversion), referred to as “residence time”. Accordingly, in some embodiments, the residence time (seconds) of the solution comprising the aza-bicyclic compound of Formula 1, or salt thereof, prior to quenching of the solution, is at least about 0.01, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 seconds. In some embodiments, the residence time (seconds) of the solution comprising the aza-bicyclic compound of Formula 1, or salt thereof, prior to quenching of the solution, is less than about 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.01 seconds. In some embodiments, the residence time (seconds) of the solution comprising the aza-bicyclic compound of Formula 1, or salt thereof, prior to quenching of the solution, is in a range provided by any two of the previously described upper and/or lower amounts, for example, between about 0.01 and 30, 1 and 29, 2 and 28, 3 and 27, 4 and 26, 5 and 25, 6 and 24, 7 and 23, 8 and 22, 9 and 21, 10 and 20, 11 and 19, 12 and 18, 13 and 17, or 14 and 16 seconds. In some embodiments, the residence time (seconds) of the solution comprising the aza-bicyclic compound of Formula 1, or salt thereof, prior to quenching of the solution, is in a range provided by any two of the previously described upper and/or lower amounts, for example, between about 1 and 10, 2 and 8, 3 and 6, or 4 and 5 seconds. In one example, the residence time (seconds) of the solution comprising the aza-bicyclic compound of Formula 1, or salt thereof, prior to quenching of the solution, is about 2.5 seconds or less. In one example, the residence time (seconds) of the solution comprising the aza-bicyclic compound of Formula 1, or salt thereof, prior to quenching of the solution, is about 5 seconds. In one example, the residence time (seconds) of the solution comprising the aza-bicyclic compound of Formula 1, or salt thereof, prior to quenching of the solution, is about 4 seconds.

It may be necessary to configure the temperature of the continuous flow reactor throughout the quenching of the solution comprising the aza-bicyclic compound of Formula 1, or salt thereof, particularly to, for example, control any exothermic reaction.

In some embodiments, the quenching of the solution comprising the aza-bicyclic compound of Formula 1, or salt thereof is performed at a temperature of less than about 20° C., about 10° C., about 0° C., about 10° C., about −20° C., about −30° C., about −40° C., about −50° C., about −60° C., about −70° C., or about −80° C. In some embodiments, the quenching of the solution comprising the aza-bicyclic compound of Formula 1 is performed at a temperature of at least about −80° C., about −70° C., about −60° C., about −50° C., about −40° C., about −30° C., about −20° C., about −10° C., about 0° C., about 10° C., or about 20° C. In some embodiments, the quenching of the solution comprising the aza-bicyclic compound of Formula 1 is performed at a temperature in a range provided by any two of the previously described upper and/or lower amounts, for example between about −80° C. and 20° C., about −70° C. and 10° C., about −60° C. and 0° C., about −50° C. and −10° C., or about −30° C. and −10° C. In some embodiments, the quenching of the solution comprising the aza-bicyclic compound of Formula 1 is performed at a temperature of about 0° C. A person skilled in the art will appreciate that the temperature at which the quenching of the solution comprising the aza-bicyclic compound of Formula 1 or salt thereof is performed, may be different to that at which the organolithium reaction or any other stage of the continuous flow process is performed. For example, the organolithium reaction may be performed at a temperature in a range of between about −50° C. and about −70° C., and the temperature at which the quenching of the solution comprising the aza-bicyclic compound of Formula 1 or salt thereof is performed at a temperature of about 0° C. Likewise, the temperature at which the quenching of the solution comprising the aza-bicyclic compound of Formula 1 or salt thereof is performed, may be different to the temperature at which any other segment of the continuous flow reactor is maintained.

### Continuous Flow Reactor Configurations

The continuous flow reactor may be configured in any suitable set-up so as to optimise the organolithium reaction to prepare the aza-bicyclic compound of Formula 1, as described herein.

Accordingly, in some embodiments, the continuous flow process comprises providing the reaction components, being the nortropinone compound of Formula 2, the halogenated compound of Formula 3, and the organolithium reagent, in one or more continuous flow streams, in a continuous flow reactor configured at a temperature effective for synthesising the aza-bicylic compound of Formula 1, or salt thereof.

That is, in one example, the nortropinone compound of Formula 2, the halogenated compound of Formula 3, and the organolithium reagent are introduced sequentially as streams into a continuous flow reactor. In such an instance, provided are separate, pre-prepared premixtures of the nortropinone compound of Formula 2, the halogenated compound of Formula 3, and the organolithium reagent, in suitable solvent(s), prior to their sequential introduction into the continuous flow reactor. The continuous flow reactor can allow for control of reaction variables, such as, for example, order of addition, concentration, flow rate, pressure, and temperature, so as to be effective for synthesising the aza-bicylic compound of Formula 1, or salt thereof.

Alternatively, more than one reaction component may be provided in each flow stream. It will therefore be appreciated that in such an instance it is necessary to provide a pre-prepared premixture of two or more of the nortropinone compound of Formula 2, the halogenated compound of Formula 3, or the organolithium reagent.

Accordingly, in some embodiments, the nortropinone compound of Formula 2 and the halogenated compound of Formula 3 are initially combined. This combining of the nortropinone compound of Formula 2 and the halogenated compound of Formula 3 may be undertaken by any suitable means as would be appreciated by the person skilled in the art. For example, the nortropinone compound of Formula 2 and the halogenated compound of Formula 3 may be initially combined in a suitable solvent prior to being introduced into the continuous flow reactor (e.g., in an external vessel and introduced in the same flow stream into the continuous flow reactor). In another example, the nortropinone compound of Formula 2 and the halogenated compound of Formula 3 may be combined following introduction to the continuous flow reactor (e.g. introduced into the continuous flow reactor in separate flow streams). In such an instance, the continuous flow reactor may be configured such that a flow stream comprising the nortropinone compound of Formula 2 is mixed with a separate flow stream comprising the halogenated compound of Formula 3, so as to produce a flow stream in the continuous flow reactor comprising the combination of the nortropinone compound of Formula 2 and the halogenated compound of Formula 3.

Regardless of the method as to how the nortropinone compound of Formula 2 and the halogenated compound of Formula 3 are combined, as discussed above, in some embodiments, a combination of the nortropinone compound of Formula 2 and the halogenated compound of Formula 3, is subsequently combined with the organolithium reagent. That is, the nortropinone compound of Formula 2 and the halogenated compound of Formula 3 are introduced into the reactor to provide a single flow stream comprising the combination of the nortropinone compound of Formula 2 and the halogenated compound of Formula 3, in which this single flow stream is then combined with the organolithium reagent. In one example, the organolithium reagent is introduced into the continuous flow reactor in a separate flow stream. This separate flow stream comprising the organolithium reagent can then be provided to react with a single flow stream comprising the combination of the nortropinone compound of Formula 2 and the halogenated compound of Formula 3. Accordingly, in some embodiments, the process comprises combining a continuous flow stream comprising the compound of Formula 2 and the compound of Formula 3, with a continuous flow stream comprising the organolithium reagent, to obtain a continuous flow stream comprising an aza-bicyclic compound of Formula 1, or salt thereof.

In some embodiments, the process comprises combining a continuous flow stream comprising the compound of Formula 2 with a continuous flow stream comprising the compound of Formula 3, to obtain a continuous flow stream comprising the compound of Formula 2 and the compound of Formula 3; and combining the continuous flow stream comprising the compound of Formula 2 and the compound of Formula 3, with a continuous flow stream comprising the organolithium reagent, to obtain a continuous flow stream comprising an aza-bicyclic compound of Formula 1, or salt thereof.

In some embodiments, the process comprises combining a continuous flow stream comprising a compound of Formula 3 with a continuous flow stream comprising an organolithium reagent, to obtain a continuous flow stream comprising an organolithium reaction intermediate; and combining the continuous flow stream comprising the organolithium reaction intermediate with a continuous flow stream comprising a compound of Formula 2, to obtain a continuous flow stream comprising an aza-bicyclic compound of Formula 1, or salt thereof.

It will be appreciated that the continuous flow reactor may be configured, and particularly flow streams provided, in any suitable means so as to allow the organolithium reaction of the nortropinone compound of Formula 2 with the halogenated compound of Formula 3 in the presence of an organolithium reagent. This includes any pre-mixing steps to provide suitable combinations of reactants/reagents. The ultimate product of the continuous flow reaction, produced via a single flow stream, is a solution comprising the aza-bicyclic compound of Formula 1, or salt thereof.

In some embodiments, the flow rate of the flow stream comprising the combination of a compound of Formula 2 and a compound of Formula 3 is configured so as to allow for optimal reaction conditions. In particular, in some instances, it may be beneficial to have different flow rates for separate flow streams, such that when the separate flow streams are combined, an overall higher flow rate is achieved. Accordingly, in some embodiments, the continuous flow stream comprising the compound of Formula 2 and compound of Formula 3 has a flow rate (mL/min) of at least about 0.5, 1.0, 1.5, 2.0, 2.5, 3.0, 3.5, 4.0, 4.5, or 5.0 L/min. In some embodiments, the continuous flow stream comprising the compound of Formula 2 and compound of Formula 3 has a flow rate (mL/min) of less than about 5.0, 4.5, 4.0, 3.5, 3.0, 2.5, 2.0, 1.5, 1.0, or 0.5 mL/min. In some embodiments, the continuous flow stream comprising the compound of Formula 2 and compound of Formula 3 has a flow rate in a range provided by any two of the previously described upper and/or lower amounts, for example, a flow rate (mL/min) between about 0.1 and 5, 0.5 and 4.5, 1.0 and 4.0, 1.5 and 3.5, or 2.0 and 3.0 mL/min.

Similarly, the temperature of the continuous flow reactor may be configured at any particular point for optimal reaction conditions. The configuration of the temperature may be relevant to, for example, increasing reagent solubility, controlling exothermic reactions, and/or increasing yield of the aza-bicyclic compound of Formula 1. The temperature configuration of the continuous flow reactor may be such that a particular segment of the continuous flow reactor is configured at a temperature different to that of at least one other segment of the continuous flow reactor. Moreover, the temperature configuration may be such that a particular stage of the continuous flow process is configured at a temperature different to that of at least one other stage of the continuous flow process. For example, the organolithium reaction and the quenching reaction may be performed at different temperatures. Accordingly, in one example, a segment of the continuous flow reactor is configured at a temperature different to that of at least one other segment of the continuous flow reactor. Similarly, in one example, a stage of the continuous flow process is configured at a temperature different to that of at least one other stage of the continuous flow process. Accordingly, in some embodiments, at any stage during the continuous flow reaction as described herein, the continuous flow reactor is configured at a temperature of less than about 20° C., about 10° C., about 0° C., about −10° C., about −20° C., about −30° C., about −40° C., about −50° C., about −60° C., about −70° C., or about −80° C. In some embodiments, at any stage during the continuous flow reaction as described herein, the continuous flow reactor is configured at a temperature of at least about −80° C., about −70° C., about −60° C., about −50° C., about −40° C., about −30° C., about −20° C., about −10° C., about 0° C., about 10° C., or about 20° C. In some embodiments, at any stage during the continuous flow reaction as described herein, the continuous flow reactor is configured at a temperature in a range provided by any two of the previously described upper and/or lower amounts, for example, between about −80° C. and 20° C., about −70° C. and 10° C., about −60° C. and 0° C., about −50° C. and −10° C., or about −30° C. and −10° C.

In some embodiments, at any stage during the continuous flow process as described herein, the segments of the continuous flow reactor are independently configured at a temperature of less than about 20° C., about 10° C., about 0° C., about −10° C., about −20° C., about −30° C., about −40° C., about −50° C., about −60° C., about −70° C., or about −80° C. In some embodiments, at any stage during the continuous flow process as described herein, the segments of the continuous flow reactor are independently configured at a temperature of at least about −80° C., about −70° C., about −60° C., about −50° C., about −40° C., about −30° C., about −20° C., about −10° C., about 0° C., about 10° C., or about 20° C. In some embodiments, at any stage during the continuous flow process as described herein, the segments of the continuous flow reactor are independently configured at a temperature in a range provided by any two of the previously described upper and/or lower amounts, for example, between about −80° C. and 20° C., about −70° C. and 10° C., about −60° C. and 0° C., about −50° C. and −10° C., or about −30° C. and −10° C.

In one example, the continuous flow process is performed at a temperature of less than about 20° C., about 10° C., about 0° C., about 10° C., about −20° C., about −30° C., about −40° C., about −50° C., about −60° C., about −70° C., or about −80° C. In one example, the continuous flow process is performed at a temperature of at least about −80° C., about −70° C., about −60° C., about −50° C., about −40° C., about −30° C., about −20° C., about −10° C., about 0° C., about 10° C., or about 20° C. In one example, the continuous flow process is performed at a temperature in a range provided by any two of the previously described upper and/or lower amounts, for example, between about −80° C. and 20° C., about −70° C. and 10° C., about −60° C. and 0° C., about −50° C. and −10° C., or about −30° C. and −10° C.

In one example, the stages of the continuous flow process are independently performed at a temperature of less than about 20° C., about 10° C., about 0° C., −about 10° C., about −20° C., about −30° C., about −40° C., about −50° C., about −60° C., about −70° C., or about −80° C. In one example, the stages of the continuous flow process are independently performed at a temperature of at least about −80° C., about −70° C., about −60° C., about −50° C., about −40° C., about −30° C., about −20° C., about −10° C., about 0° C., about 10° C., or about 20° C. In one example, the stages of the continuous flow process are independently performed at a temperature in a range provided by any two of the previously described upper and/or lower amounts, for example, between about −80° C. and 20° C., about −70° C. and 10° C., about −60° C. and 0° C., about −50° C. and −10° C., or about −30° C. and −10° C.

In one example, the organolithium reaction is performed at a temperature of less than about 20° C., about 10° C., about 0° C., about 10° C., about −20° C., about −30° C., about −40° C., about −50° C., about −60° C., about −70° C., or about −80° C. In one example, the organolithium reaction is performed at a temperature of at least about −80° C., about −70° C., about −60° C., about −50° C., about −40° C., about −30° C., about −20° C., about −10° C., about 0° C., about 10° C., or about 20° C. In one example, the organolithium reaction is performed at a temperature in a range provided by any two of the previously described upper and/or lower amounts, for example, between about −80° C. and 20° C., about −70° C. and 10° C., about −60° C. and 0° C., about −50° C. and −10° C., or about −30° C. and −10° C.

### Solvent

A person skilled in the art will appreciate that a variety of suitable solvents may be employed for the continuous flow process. In some embodiments, the continuous flow process is performed in a polar solvent, such as a polar aprotic solvent. In some embodiments, the continuous flow process is performed in a non-polar solvent, such as a non-polar aprotic solvent. Examples of polar aprotic solvents include, but are not limited to, halogenated hydrocarbons, nitriles, esters, carbonate esters, ethers, sulfoxides, sulfones, amides, nitroalkanes, and pyrrolidines. Examples of ketones include, but are not limited to, acetone, methylethyl ketone (MEK), methylbutyl ketone (MBK), methylisobutyl ketone (MIBK), and methylisopropyl ketone. Examples of nitriles include, but are not limited to, acetonitrile (MeCN). Examples of esters include, but are not limited to, ethyl formate, methyl acetate (MeOAc), ethyl acetate (EtOAc), propyl acetate, isopropyl acetate (iPAC), n-butyl acetate, and isobutyl acetate. Examples of carbonate esters include, but are not limited to, dimethyl carbonate (DMC) and propylene carbonate (PC). Examples of polar and non-polar ethers include, but are not limited to, methyl-tert-butyl ether (MTBE), diethyl ether, 1,4-dioxane, 2-methoxyethanol, 2-ethoxyethanol, dimethoxyethane (DME or monoglyme), 1,1-dimethoxymethane, 2,2-dimethoxypropane, 1,1-diethoxypropane, isopropyl ether, petroleum ether, cyclopentyl methyl ether (CPME), anisole (methoxybenzene), methyltetrahydrofuran (MeTHF), and tetrahydrofuran (THF). Examples of sulfoxides include, but are not limited to, dimethylsulfoxide (DMSO). Examples of sulfones include, but are not limited to, sulfolane. Examples of amides include, but are not limited to, formamide, N,N-dimethylacetamide, and N,N-dimethylformamide (DMF). Examples of nitroalkanes include, but are not limited to, nitromethane. Examples of pyrrolidines include, but are not limited to, N-methylpyrrolidone (NMP). Examples of non-polar aprotic solvents include benzene, chloroform, cyclohexane, diethyl ether, hexane, pentane, and toluene. In one example, the non-polar aprotic solvent is toluene. Examples of polar and non-polar halogenated hydrocarbons, such as chlorocarbons, include, but are not limited to, 1,1,1-trichloroethane, 1,1-dichloroethene, and 1,2-dichloroethene. In some embodiments, the solvent comprises or consists of one or more polar aprotic ether solvents, such as MeTHF and THF. In one example, the continuous flow process is performed in an ether, such as MeTHF or THF. In one example, the continuous flow process is performed in MeTHF. In one example, the continuous flow process is performed in THF. The solvent may be present in the continuous flow process in any amount suitable so as to effect the organolithium reaction. In some examples, the solvent may be anhydrous. For example, the amount of water in the solvent may be less than about (in ppm) 500, 400, 300, 200 100, 75, 50, 25, 10, 5, or 1 ppm.

### Dilution Streams

At any stage during the continuous flow reaction, it may be necessary to dilute one or more flow streams. It will be appreciated that diluting a flow stream will subsequently reduce the concentration of any reactants/reagents in that flow stream. This may be necessary as a way of optimising the reaction. Accordingly, in some embodiments, the continuous flow process further comprises combining a continuous flow stream comprising a solvent with a continuous flow stream comprising the organolithium reagent, to controllably dilute the concentration of one or more reagents. In one example, the continuous flow process further comprises combining a continuous flow stream comprising a solvent with a continuous flow stream comprising the organolithium reagent, to controllably dilute the concentration of the organolithium reagent.

Furthermore, a dilution stream may be utilised to modify the solvent composition at the point of the reaction so as to optimise that reaction. For example, introducing a second solvent through a dilution stream may increase or decrease miscibility, therefore increasing or decreasing reagent solubility, and therefore potentially increasing or decreasing reaction rate and conversion of starting materials and reagents to the reaction product. As used herein, the term “miscibility” refers to the property of two substances (e.g., solvents) to mix in all proportions to form a homogeneous mixture (e.g., a solution). In one example, the continuous flow process further comprises combining a continuous flow stream comprising a first solvent with a continuous flow stream comprising a second solvent, wherein the second solvent acts to increase the resultant miscibility. In one example, the continuous flow process further comprises combining a continuous flow stream comprising a first solvent with a continuous flow stream comprising a second solvent, wherein the second solvent acts to decrease the resultant miscibility.

Advantageously, the use of a continuous flow reactor allows for the introduction of a dilution stream at any suitable point in the reaction.

In some embodiments, the dilution stream is provided with a flow rate (mL/min) of at least about 0.1, 0.5, 1, 2, 3, 4, 5, 6, 7, 8, 9, or 10 L/min. In some embodiments, the continuous flow stream comprising the solvent is provided with a flow rate (mL/min) of less than about 10, 9, 8, 7, 6, 5, 4, 3, 2, 1, 0.5, or 0.1 mL/min. In some embodiments, the continuous flow stream comprising solvent is in a range provided by any two of the previously described upper and/or lower amounts, for example, a flow rate (mL/min) of between about 0.1 and 10, 1 and 9, or 5 and 7 mL/min.

### Yield

As used herein, the term “yield” will be taken to mean the amount of either crude or purified compound (e.g., an aza-bicyclic compound of Formula 1) obtained from a reaction, measured as a percentage of theoretical yield of the compound in that reaction, as would be understood by the person skilled in the art. Accordingly, in some embodiments, the continuous flow process provides a yield of an aza-bicyclic compound of Formula 1 of at least 20%, at least 30%, at least 40%, at least 50%, at least 60%, at least 70%, at least 80%, or at least 90% as determined from a compound of Formula 2 or a compound of Formula 3 as starting materials. Similarly, in some embodiments, the continuous flow process provides a yield of an aza-bicyclic compound of Formula 1 of between about 20% and 90%, between about 30% and 80%, between about 50% and 80%, or between about 50% and 70%, or between about 60% and 80%, or between about 70% and 80%, as determined from a compound of Formula 2 or a compound of Formula 3 as starting materials.

The continuous flow process may be monitored by any conventional means as would be known by the person skilled in the art, for example, TLC (thin-layer chromatography) or HPLC (high performance liquid chromatography). Advantageously, the ability to monitor the continuous flow process provides an indication of the consumption (conversion) of the starting materials (i.e., a compound of Formula 2 and/or a compound of Formula 3). Accordingly, in some embodiments, the continuous flow process provides a consumption of a compound of Formula 3 or a compound of Formula 2 of at least 20%, at least 30%, at least 40%, at least 50%, at least 60%, at least 70%, at least 80%, or at least 90%, as measured by HPLC. In some embodiments, the continuous flow process provides a consumption of a compound of Formula 3 or a compound of Formula 2 of between about 20% and 90%, between about 30% and 80%, between about 50% and 80%, or between about 60% and 80%, as measured by HPLC. It will be understood that the consumption of a starting material (i.e., a compound of Formula 2 and/or a compound of Formula 3) may be measured at any point during the reaction. Typically, an aliquot of the reaction mixture will be subject to HPLC, where the relevant component peaks are identified and integrated relative to one another.

### Productivity

As used herein, the term “productivity” refers to the rate of product output from the continuous flow reactor, as measured in grams per hour. Accordingly, in some embodiments, the continuous flow process provides a productivity rate, or rate of product output (in grams per hour), of at least about 0.1, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 500, 1000, 2000, 5000 g/h. In some embodiments, the continuous flow process has a productivity rate, or rate of product output (in g/h), in a range provided any two of the previously described amounts, for example a product output (in g/h) of between about 0.1 and 100, 1 and 50, 5 and 40, 10 and 30, or 15 and 25 g/h.

### Space-Time Yield

As used herein, the term “space-time yield” refers to the amount of product obtained per one hour for one litre of reactor volume (in kilograms per litre per hour). Accordingly, in some embodiments, the continuous flow process has a space-time yield (in kg/L/h) of at least about 1, 5, 10, 20, 30, 40, 50, 60, 70, 80, 90, 100, 200, 400, 600, 800, 1000, 1500, 2000, 2500, 3000, 4000, 5000, 10000 kg/L/h. In some embodiments, the continuous flow process has a space-time yield (in kg/L/h) in a range provided by any two of the previously described amounts, for example, a space-time yield (in kg/L/h) of between about 1 and 10000, 5 and 200, 10 and 100, 20 and 90, 30 and 80, or 40 and 60 kg/L/h.

### Reactor Segments

The continuous flow reactor may be defined by various “segments” through which the flow streams pass. Indeed, the continuous flow process as described herein may be considered to be conducted in one or more segments within the continuous flow process. A segment may comprise a combination of one or more conduits or portions thereof, wherein all conduits or portions of the combination are collectively adjoining. Such segments may assist in defining particular configurations within the reactor, for example, concentration, solvent, and temperature. As such, the total volume of each segment may be independently defined, and will be referred to as “segment volume” (in mL). Accordingly, in one example, the continuous flow process has a total segment volume (mL) of at least about 0.01, 0.05, 0.1, 0.5, 1, 2, 3, 4, 5, 6, 7, 8, 9, 10, 20, 30, 40, 50, 60, 70, 80, 90, 100, 500, or 1000 mL. In some embodiments, the continuous flow process has a total segment volume (mL) in a range provided by any two of the previously described amounts, for example, a total segment volume (mL) of between about 1 and 1000, 5 and 1000, 100 and 1000, or 500 and 1000 mL.

### Purity

As used herein, the term “purity” refers to the amount of the desired substance relative to the total amount of all substances found in a sample, such as impurities (or undesired substances) and optionally, any solvents that may be present in the sample. Purity may be expressed qualitatively (e.g. high purity), or expressed quantitatively (e.g. as a percentage). Purity may be assessed based upon weight, volume or molar ratios of one substance relative to the others. It will therefore be appreciated that a sample of high purity of the desired substance, for example, the aza-bicyclic compound of Formula 1, is optimal. Accordingly, in some embodiments, the process described herein provides an aza-bicyclic compound of Formula 1 in high purity. As would be understood by a skilled person, purity is a measure independent of yield. That is, a sample may have a high purity, albeit a low yield. As used herein, the term “high purity” refers to at least 70% of the ultimately obtained sample being the desired compound (e.g., the aza-bicyclic compound of Formula 1), which may be measured, for example, by HPLC or NMR methods. The purity of a sample may be measured based on the crude reaction mixture, the sample isolated from the reaction mixture (i.e., following the reaction work-up), or the purified sample (i.e., following chromatography, recrystallization, etc.).

In some embodiments, the continuous flow process provides an aza-bicyclic compound of Formula 1 in at least 30%, 40%, 50%, 60%, 70%, 80%, 85%, 90%, or 95% purity.

### Continuous Flow Reactor System

It will be appreciated that there are numerous variations of both research and industrial/commercial continuous flow reactor systems. Specific variations may lend themselves to greater utility when performing the continuous flow process, as described herein. The flow reactor should provide sufficient heat transfer and mass transfer to maintain the desired level of selectivity towards production of the compound of Formula 1. These parameters are dependent on the flow rate input streams, but can be enhanced by an optimised reactor design. For example, use of static mixing elements in a plug flow reactor will improve mass transfer. Examples of continuous flow reactor systems include, but are not limited to, plug flow reactors, tubular reactors, fixed bed reactors, fluid bed reactors, continuous stirred tank reactors (CSTR), spinning disk reactors, spinning tube reactors, multi-cell flow reactors, oscillatory flow reactors, microreactors, hex reactors, and aspirator reactors. In one example, the continuous flow process described herein is performed in a continuous flow reactor system selected from the group consisting of a plug flow reactor, tubular reactor, fixed bed reactor, fluid bed reactor, continuous stirred tank reactor (CSTR), spinning disk reactor, spinning tube reactor, multi-cell flow reactor, oscillatory flow reactor, microreactor, hex reactor, and aspirator reactor. In one example, the continuous flow process described herein is performed using a plug flow reactor. In one example, the continuous flow process described herein is performed using tubular reactor. In one example, the continuous flow process described herein is performed using a fixed bed reactor. In one example, the continuous flow process described herein is performed using a fluid bed reactor. In one example, the continuous flow process described herein is performed using continuous stirred tank reactor (CSTR). In one example, the continuous flow process described herein is performed using spinning disk reactor. In one example, the continuous flow process described herein is performed using a spinning tube reactor. In one example, the continuous flow process described herein is performed using multi-cell flow reactor. In one example, the continuous flow process described herein is performed using an oscillatory flow reactor. In one example, the continuous flow process described herein is performed using a microreactor. In one example, the continuous flow process described herein is performed using a hex reactor. In one example, the continuous flow process described herein is performed using an aspirator reactor.

The plug flow reactor (PFR), sometimes called continuous tubular reactor (CTR) or piston flow reactor, is a reactor used to perform and describe chemical reactions in continuous, flowing systems. The PFR reactor model is used to predict the behaviour of chemical reactors of such design, so that key reactor variables, such as the dimensions of the reactor, can be estimated. Fluid going through a PFR may be modelled as flowing through the reactor as a series of infinitely thin coherent “plugs”, each with a uniform composition, traveling in the axial direction of the reactor, with each plug having a different composition from the ones before and after it. The key assumption is that as a plug flows through a PFR, the fluid is perfectly mixed in the radial direction (i.e. in the lateral direction) but not in the axial direction (forwards or backwards).

In one example, the continuous flow process described herein is performed using a tubular reactor. In one example, the continuous flow process described herein is performed in a continuous stirred tank reactor (CSTR). In one example, the continuous flow process described herein is performed in a microreactor.

Accordingly, the terms used herein to define the reactor type used in the context of the invention such like “continuous flow reactor”, “plug flow reactor”, “tubular reactor”, “continuous flow reactor system”, “plug flow reactor system”, “tubular reactor system”, “continuous flow system”, “plug flow system”, “tubular system”, “microreactor” are synonymous to each other and interchangeable with each other.

The reactor or system may be arranged as a multitude of conduits, which may be, for example, linear, looped, meandering, circled, coiled, or combinations thereof. If coiled, for example, then the reactor or system is also called a “coiled reactor” or “coiled system”.

The conduits that comprise the multitude of conduits, may be described in terms of their maximum inner diameter or inner cross-section dimension (i.e. the radial or lateral dimension, respectively). Such a description defines the lateral dimension of the reactor or reactor system. In some embodiments, the lateral dimension of the reactor or reactor system may be less than about 10 cm, less than about 9 cm, less than about 8 cm, less than about 7 cm, less than about 6 cm, less than about 5 cm, less than about 4 cm, less than about 3 cm, less than about 2 cm, less than about 1 cm, less than about 0.9 cm, less than about 0.8 cm, less than about 0.7 cm, less than about 0.6 cm, less than about 0.5 cm, less than about 0.4 cm, less than about 0.3 cm, less than about 0.2 cm, less than about 10 mm, less than about 9.5 mm, less than about 9.0 mm, less than about 8.5 mm, less than about 8.0 mm, less than about 7.5 mm, less than about 7.0 mm, less than about 6.5 mm, less than about 6.0 mm, less than about 5.5 mm, less than about 5.0 mm, less than about 4.5 mm, less than about 4.0 mm, less than about 3.5 mm, less than about 3.0 mm, less than about 2.5 mm, less than about 2.0 mm, less than about 1.5 mm, less than about 1.0 mm, less than about 0.9 mm, less than about 0.8 mm, less than about 0.7 mm, less than about 0.6 mm, less than about 0.5 mm, less than about 0.4 mm, less than about 0.3 mm, less than about 0.2 mm, or less than about 0.1 mm. In some embodiments, the lateral dimension of the reactor or reactor system may be greater than about 10 cm, greater than about 9 cm, greater than about 8 cm, greater than about 7 cm, greater than about 6 cm, greater than about 5 cm, greater than about 4 cm, greater than about 3 cm, greater than about 2 cm, greater than about 1 cm, greater than about 0.9 cm, greater than about 0.8 cm, greater than about 0.7 cm, greater than about 0.6 cm, greater than about 0.5 cm, greater than about 0.4 cm, greater than about 0.3 cm, greater than about 0.2 cm, greater than about 10 mm, greater than about 9.5 mm, greater than about 9.0 mm, greater than about 8.5 mm, greater than about 8.0 mm, greater than about 7.5 mm, greater than about 7.0 mm, greater than about 6.5 mm, greater than about 6.0 mm, greater than about 5.5 mm, greater than about 5.0 mm, greater than about 4.5 mm, greater than about 4.0 mm, greater than about 3.5 mm, greater than about 3.0 mm, greater than about 2.5 mm, greater than about 2.0 mm, greater than about 1.5 mm, greater than about 1.0 mm, greater than about 0.9 mm, greater than about 0.8 mm, greater than about 0.7 mm, greater than about 0.6 mm, greater than about 0.5 mm, greater than about 0.4 mm, greater than about 0.3 mm, greater than about 0.2 mm, or greater than about 0.1 mm. In some embodiments, the lateral dimension of the reactor or reactor system, is in a range provided by any two of the above upper and/or lower amounts, for example, wherein the lateral dimension is between about 0.1 mm to 1 cm, 0.1 mm to 0.5 mm, 1 mm to 1 cm, or 1 cm to 3 cm.

If the largest lateral dimension of a reactor or reactor system is less than about 1 mm, then the reactor is typically referred to as a “microreactor”. Thus, in when utilising a microreactor, in one example, the lateral dimension of the reactor or reactor system may be in the range of from about 0.1 mm up to about 1 mm.

The reactor or reactor system may be configured such that, in use, the pressure drop across the reactor (in Pa/m) is in a range of about 0.1 to 1,000,000 Pa/m (or 1 MPa/m), including at any value or range of any values there between. For example, the pressure drop across the continuous flow reactor (in Pa/m) may be less than about 500,000, 250,000, 100,000, 50,000, 10,000, 5,000, 1,000, 750, 500, 250, 100, 75, 50, 25, 20, 15, 10, or 5 Pa/m. Alternatively, total pressure drop across the reactor may be measured in MPa, and is in a range of about 0 to 10 MPa (or 100 bar), including at any value or range of any values there between. For example, the total pressure drop across the continuous flow reactor (in MPa) may be less than about 10, 5, 4, 3, 2, 1, 0.9, 0.8, 0.7, 0.6, 0.5, 0.4, 0.3, 0.2, 0.1, 0.05, 0.04, 0.03, 0.02, or 0.01 MPa. The continuous flow reactor may be configured to provide a lower pressure drop relative to a specific flow rate. In this regard, reactor, system, and processes, as described herein, may be provided with parameters suitable for industrial application. The above pressure drops may be maintained where the volumetric flow rate is at least 0.1, 0.5, 1, 1.5, 2, 2.5, 3, 3.5, 4, 4.5, 5, 6, 7, 8, 9, 10, 20, 30, 40, 50 mL/min.

### Scale-Up of Continuous Flow Process

As will be appreciated by the person skilled in the art, the utilisation of a continuous flow reactor enables for continuous input/output of material, meaning scalability of the continuous flow process is achievable (e.g., by continuing to run the continuous flow process for a longer time period, by using reactors with larger lengths or cross sectional areas which allow the use of higher flow rates). In one example, the continuous flow process described herein is performed in a microreactor. In an alternative embodiment of the invention, it is also optionally desired to employ a continuous flow reactor other than a microreactor, particularly in the instance where greater production is desirable (i.e., scale-up). In such a case, it is desirable to employ a continuous flow reactor with lateral dimensions that are greater than that indicated above for a microreactor. Accordingly, in the instance of employing a larger continuous flow reactor (i.e., scale-up), the term “continuous flow reactor” denotes a device in which chemical reactions take place in a confinement with typical lateral dimensions that are greater than approximately 1 mm, and may be up to about 5 mm, 10 mm, 2 cm, 3 cm, 4 cm, 5 cm, 6 cm, 7 cm, 8 cm, 9 cm, or even 10 cm. In such embodiments, it may be desirable to employ as a continuous flow reactor a plug flow reactor and/or a tubular flow reactor. When employing a larger continuous flow reactor, consideration of the capability of the reactor to facilitate sufficient heat transfer and mass transfer must be undertaken, in order to retain the desired level of selectivity towards production of the compound of Formula 1. As will be appreciated by the person skilled in the art, it may be desirable for the reactor to allow reagents to rapidly mix, whilst maintaining a low reaction temperature. As will further be appreciated, the transfer of heat and/or mass facilitated by a reactor, may be tuned through the inclusion of mixing elements, such as static mixers.

During scale-up of the continuous flow process as described herein, it may be desirable to employ higher flow rates for the continuous flow streams in the continuous flow process. For example, such higher flow rates are up to about 2 times higher, up to about 5 times higher, up to about 10 times higher, up to about 100 times higher, up to about 500 times higher, up to about 1000 times higher, or any intermediate flow rate of from about 1 up to about 1000 times higher, of from about 1 up to about 500 times higher, of from about 1 up to about 100 times higher, of from about 1 up to about 10 times higher, of from about 1 up to about 5 times higher, or of from about 1 up to about 2 times higher, each as compared to the typical flow rates described herein (i.e., for a microreactor). In such embodiments, it may be desirable to adjust independently as described herein, the flow rates of the continuous flow streams that comprise the compound of Formula 1, the compound of Formula 2, the organolithium reagent, and/or the quenching solution.

Furthermore, during scale-up, it may be desirable to employ longer residence times in the continuous flow reactor. For example, such longer residence times, are up to about 2 times longer, up to about 3 times longer, up to about 4 times longer, up to about 5 times longer, up to about 6 times longer, up to about 7 times longer, or any intermediate flow rate of from about 1 up to about 7 times longer, of from about 1 up to about 6 times longer, of from about 1 up to about 5 times longer, of from about 1 up to about 4 times longer, of from about 1 up to about 3 times longer, or of from about 1 up to about 2 times longer, each as compared to the typical residence times indicated herein (i.e., for a microreactor). In some embodiments, it may be desirable to employ shorter residence times in the continuous flow reactor. For example, such shorter residence times, are up to about 2 times shorter, up to about 3 times shorter, up to about 4 times shorter, up to about 5 times shorter, up to about 6 times shorter, up to about 7 times shorter, or any intermediate flow rate of from about 1 up to about 7 times shorter, of from about 1 up to about 6 times shorter, of from about 1 up to about 5 times shorter, of from about 1 up to about 4 times shorter, of from about 1 up to about 3 times shorter, or of from about 1 up to about 2 times shorter, each as compared to the typical residence times indicated herein (i.e., for a microreactor). In such embodiments, it may be desirable to adjust independently as described herein, the residence time of each segment of the continuous flow reactor.

It is of course possible to use one or more reactors, in the production, preferably for the industrial production, of the compounds described herein. If more than one reactor is used in the production, preferably for the industrial production, then these reactors can be used in parallel and/or subsequent arrangements. For example, two, three, four, or more reactors can be used in parallel and/or subsequent arrangements. A person skilled in the art will appreciate that the continuous flow process described herein may utilise multiple continuous flow reactors in parallel and/or subsequent arrangements. Utilising such a configuration of reactors may find particular utility in the scale-up of the continuous flow process, as described herein. Furthermore, the use of one or more reactors in parallel allows for overall continued production in the event operation of a particular reactor or reactors is ceased for the purpose of maintenance, cleaning, or in the event of a blockage or failure. Cleaning of a reactor may take place by flushing the reactor with one or more suitable cleaning solvents (‘cleaning-in-place’), or alternatively, following reactor shut down and disassembly. The formation of blockages can be monitored through the use of pressure sensors.

### Synthesis of Xanamem

As discussed above, the aza-bicyclic heteroaryl moieties are frequent intermediates in the synthesis of numerous pharmaceutical compounds, including Xanamem.

In the synthesis of Xanamem, it is possible that any reaction previous to, or subsequent to, the synthesis of the aza-bicyclic heteroaryl moiety, as described herein, is undertaken in a continuous flow reactor. Alternatively, in the synthesis of Xanamem, it is possible that any reaction previous to, or subsequent to, the synthesis of the aza-bicyclic heteroaryl moiety, as described herein, is undertaken via batch or semi-batch reaction conditions.

Accordingly, in some embodiments, the continuous flow process further comprises preparing a salt of an amine bicyclic compound of Formula 4:

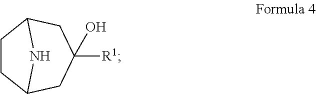

wherein the process comprises reacting an aza-bicyclic compound of Formula 1:

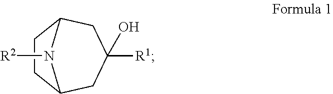

with an acid to remove the amine protecting group, to obtain a salt of the compound of Formula 4.

In some embodiments, the compound of Formula 1 is isolated prior to the reaction with an acid to remove the amine protecting group to obtain a salt of the compound of Formula 4. Isolation may occur via any suitable means as would be understood by the person skilled in the art. Alternatively, in some embodiments, the compound of Formula 1 is not isolated prior to the reaction with an acid to remove the amine protecting group to obtain a salt of the compound of Formula 4.

The acid utilised to remove the amine protecting group may be any suitable acid. In one example, the acid is para-toluenesulfonic acid. In such an instance, when the acid is para-toluenesulfonic acid, it will be appreciated that the compound of Formula 4 may be obtained as a para-toluenesulfonate salt. In one example, R² is tert-butyloxycarbonyl (Boc), and the acid utilised to remove the amine protecting group is para-toluenesulfonic acid.

In some embodiments, the continuous flow process further comprises preparing a heterocyclic methanone compound of Formula 5:

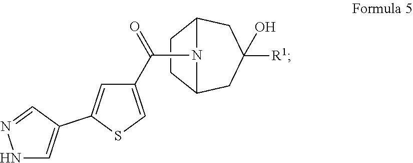

wherein the process comprises reacting a carboxylic acid compound of Formula 6, or a salt thereof:

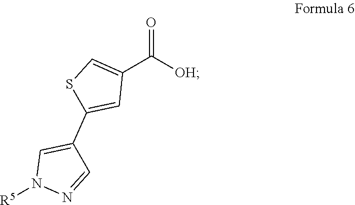

with the amine bicyclic compound of Formula 4, or salt thereof:

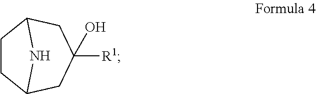

in the presence of at least one coupling reagent, to obtain the heterocyclic methanone compound of Formula 5.

In some embodiments, the continuous flow process further comprises preparing Xanamem:

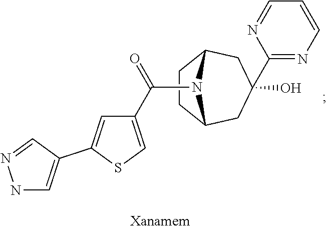 [SMILES: O=C(c1csc(-c2cn[nH]c2)c1)N1[C@@H]2CC[C@H]1C[C@](O)(c1ncccn1)C2]

wherein the process comprises reacting a carboxylic acid compound of Formula 7, or a salt thereof:

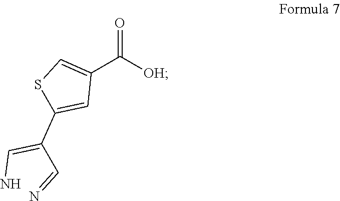 [SMILES: N=C/C(=C\N)c1cc(C(=O)O)cs1]

with the amine bicyclic compound of Formula 1ai, or salt thereof:

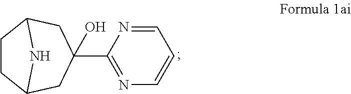 [SMILES: OC1(c2ncccn2)CC2CCC(C1)N2]

in the presence of at least one coupling reagent, to obtain Xanamem.

As would be understood by the person skilled in the art, the term “coupling reagent” refers to a compound that provides a chemical bond between two chemical moieties. In one example, the coupling reagent is an “amide coupling reagent”, and provides a chemical bond between a carboxylic acid moiety and an amine moiety, thereby forming an amide bond.

In some embodiments, the coupling reagent is an oxime coupling reagent. Such a coupling reagent may also be referred to as an “additive”. In some embodiments, the oxime coupling reagent is selected from the group consisting of OxymaPure (2-cyano-2-(hydroxyimino)acetate), K-Oxyma (potassium 2-cyano-2-(hydroxyimino)-acetate), COMU (1-[(1-(cyano-2-ethoxy-2 oxoethylideneaminooxy)dimethyl-aminomorph-olinomethylene)]methanaminium hexafluorophosphate), PyOxym-M, PyOxim (O-[(cyano(ethoxycarbonyl)-methyliden)amino]yloxytripyrrolidinophosphonium hexafluoro-phosphate), HONM (isonitroso Meldrum's acid), Ocyma-B, Oxyma-T, Amox, HMMU, and Fmoc-Amox. In one example, the oxime coupling reagent is OxymaPure (2-cyano-2-(hydroxyimino)acetate). In some embodiments, the carbodiimide coupling reagent is selected from the group consisting of DCC (dicyclohexylcarbodiimide), DIC (diisopropylcarbodiimide), EDAC·HCl (N-(3-dimethylaminopropyl)-N′-ethylcarbodiimide·HCl), and EDC (1-ethyl-3-(3-dimethylaminopropyl) carbodiimide). In one example, the carbodiimide coupling reagent is EDC (1-ethyl-3-(3-dimethylaminopropyl) carbodiimide). In some embodiments, the coupling reagent is selected from at least one oxime coupling reagent and at least one carbodiimide coupling reagent, which may each be provided according to any embodiments or examples thereof as described herein. In one example, the coupling reagent comprises OxymaPure (2-cyano-2-(hydroxyimino) acetate) and EDC (1-ethyl-3-(3-dimethylaminopropyl) carbodiimide). In one example, the coupling reagent is an oxime coupling reagent. In one example, the coupling reagent is a carbodiimide coupling reagent.

### EXAMPLES

### General Experimental Information

### Abbreviations

DIC N,N′-diisopropylcarbodiimide
DIPEA N,N′-diisopropylethylamine
DMSO Dimethyl sulfoxide
EDC 1-Ethyl-3-(3-dimethylaminopropyl)carbodiimide
HCl Hydrochloric acid
HOPO 2-Hydroxypyridine-N-oxide
HPLC High Performance Liquid Chromatography
IPA Isopropyl alcohol
IPC Ion pair chromatography
2-MeTHF 2-Methyltetrahydropyran
NMR Nuclear magnetic resonance
pTSA p-Toluenesulfonic acid
TFA Trifluoroacetic acid
THF Tetrahydrofuran
THP Tetrahydropyran
TMS Tetramethylsilane

### General Experimental Scheme

The experimental will focus on the reaction of EU1F (2-iodopyrimidine) with N-boc-nortropinone to produce EU1G, an aza-bicyclic compound of Formula 1. EU1G is a key intermediate in the synthesis of Xanamem.

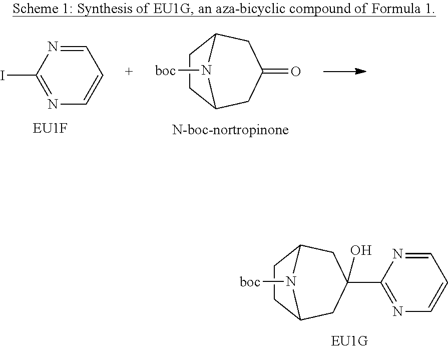

EU1F (CAS: 31462-54-1) was sourced from AA BLOCKS (catalogue: AG00C6WU; Lot: AGN22-762989-1). N-boc-nortropinone (CAS: 185099-67-6) was sourced from Apollo Scientific (catalogue: OR12337; Lot: AS498687). n-Hexyllithium (HexLi, 2.5 M in hexanes, CAS: 21369-64-2) was sourced from Thermo Scientific (catalogue: 301651000). n-Butyllithium (n-BuLi, 2.5 M in hexanes, CAS: 109-72-8) was sourced from Aldrich (catalogue: 230707). Phenyllithium (1.9 M in dibutyl ether, CAS: 591-51-5) was sourced from Aldrich (catalogue: 593230). Methyllithium (MeLi, 1.6 M in Et₂O, CAS: 917-54-4) was sourced from Sigma Aldrich (catalogue: 197343). Methyllithium lithium bromide complex (MeLi·LiBr. 1.5 M in Et₂O, CAS: 332360-06-2) was sourced from Sigma Aldrich (catalogue: 186201). p-Toluenesulfonic acid monohydrate (pTSA·H₂O, CAS: 6192-52-5) was sourced from Acros (catalogue: 13902 1000). Tetrahydrofuran (THF, CAS: 109-99-9) was sourced from Fisher Scientific (catalogue: 348450010).

Nuclear magnetic resonance (NMR) spectra were recorded on a Bruker 300 MHz instrument. ¹H and ¹³C spectra were recorded at 300 MHz and 75 MHz respectively, with a chemical shift relative to TMS expressed in parts per million (ppm). The samples were prepared in CDCl₃ or DMSO-d₆. The letters s, d, t and m are used to indicate singlet, doublet, triplet and multiplet respectively.

HPLC Method A (Polar; 12.5 min): HPLC analysis was carried out on a Shimadzu LC20 system, using a Macherey-Nagel Nucleodur C18 reversed-phase analytical column (150 mm×4.6 mm, particle size 5 m) at 37° C. using mobile phases A (90:10 v/v water/acetonitrile+0.1% TFA) and B (MeCN+0.1% TFA) at a combined flow rate of 1.5 mL/min. The following gradient was applied: start at 3% solvent B, linear increase from 3% to 5% B over 3 min, linear increase from 5% to 30% B over 4 min, linear increase from 30% to 100% B over 3 min, hold at 100% B for 2 min, linear decrease from 100% to 3% B over 0.5 min, hold at 3% B for 2.5 min. Final purity assessment of the boc deprotected pTSA salt was performed using HPLC Method A.

HPLC Method B (Standard; 10 min): HPLC analysis was carried out on a Shimadzu LC20 system, using a Macherey-Nagel Nucleodur C18 reversed-phase analytical column (150 mm×4.6 mm, particle size 5 m) at 37° C. using mobile phases A (90:10 v/v water/acetonitrile+0.1% TFA) and B (MeCN+0.1% TFA) at a flow rate of 1.5 mL/min. The following gradient was applied: start at 30% solvent B, increase (linear gradient) to 100% solvent B over 10 min, then reduce to 30% solvent B for 3 min, to prime the column for the next injection. Unless otherwise stated, all experiments were analysed using HPLC Method B.

For all flow set-ups, standard PFA tubing (0.8 mm or 1.6 mm i.d.), PTFE or PEEK fittings and T-pieces were used. For pumping reagent solutions, syringe pumps (Syrris Asia) equipped with 0.5/0.25 mL (flow rates ≤1.25 mL/min), 1.0/0.5 mL (flow rates ≤2.50 mL/min) or 5.0/2.5 mL (flow rates ≤10 mL/min) syringes were used. All pumps were used with check valves (Upchurch, CV-3321) and internal pressure sensors. For safety reasons, the pressure limit of the pumps was set to 20 bar (automatic shutoff at pressures >20 bar). All pumps were tested and found to pump with flow rates accurate within ±3% of the set value.

All flow reactions were performed in a Modular MicroReaction System (MMRS) manufactured by Ehrfeld Mikrotechnik (Wendelsheim, Germany). For the reagent inlets 1/16″ input connectors (0711-2-00224-F) were used. For the quench solution (citric acid in methanol) and the outlet ⅛″ input connectors (0711-2-0224-F) were used. A FlowPlate Lab (1701-3-0004-F) equipped with a TG design Process Plate (Art.-No. 1701-2643-HC, TG mixer, nominal width 0.2 mm, volume 0.344 mL, flow rate range 1.5-15 mL/min, channel width (max.) 0.6 mm×0.5 mm, Hastelloy C22) was used, and the temperature was controlled by a cryostat (Lauda Proline RP-890).

Feed solutions containing 2-iodopyrimidine and N-boc-nortropinone were prepared in volumetric flasks. 2-Iodopyrimidine and N-boc-nortropinone and −20 mol % of biphenyl as internal standard (only used for reaction development, and would not be included in a larger scale process) were weighed into the corresponding flask, placed under argon and brought up to volume using reaction solvent (dry THF if not stated otherwise).

For optimization experiments, all substrates and reagent streams were introduced via sample loops (PFA, 0.8 mm i.d., volume=1-5 mL, exact volumes depending on the equivalents of the reagents). This was enabled using 2-position 6-port valves (IDEX, V-450), which were configured to allow loading of reagents into the sample loop, then injection of the reagents into the reactor. By this approach, the amount of material required for flow experiments can be minimised, since pumps do not need to be individually primed with reagents. Before every experiment the whole reactor system was flushed with dry THF for at least 10 min at the maximum flow rates of the used syringe pumps.

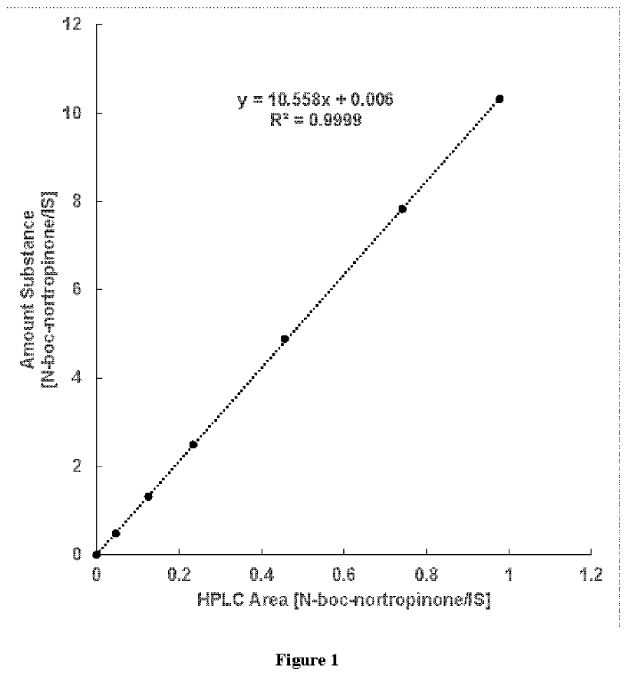

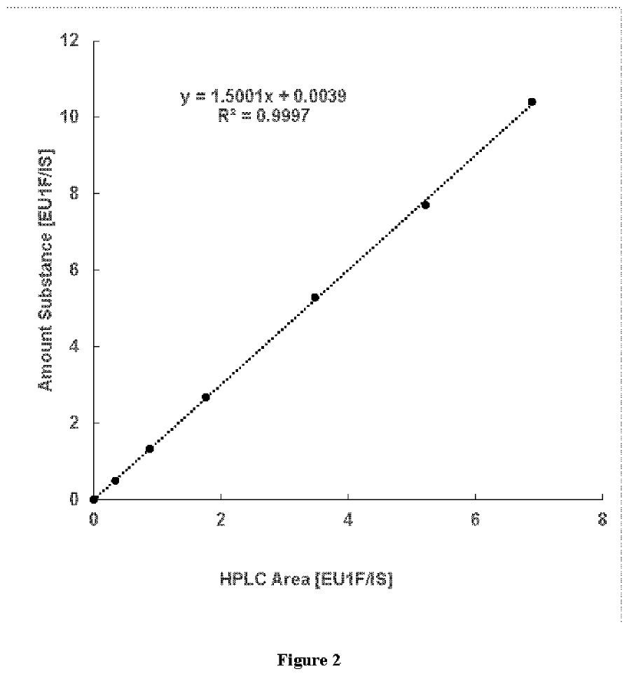

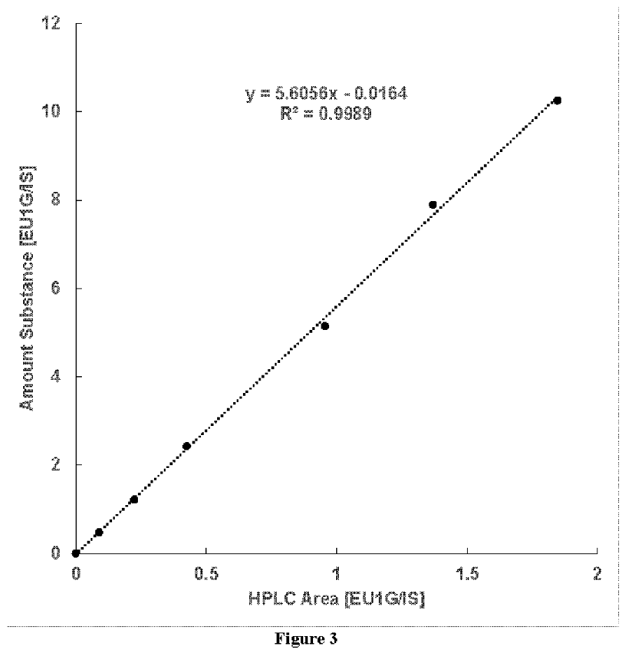

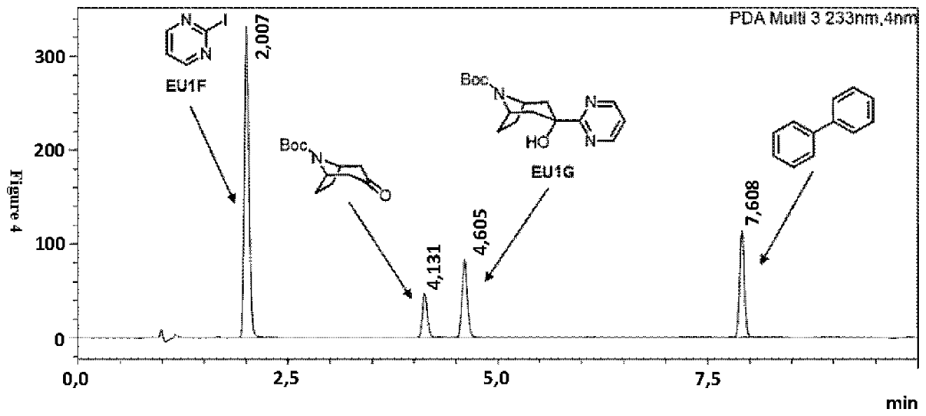

Collected solutions after reaction (and citric acid quench) were analysed using calibrated HPLC analysis to determine amounts of 2-iodopyrimidine, EU1G and N-boc-nortropinone. HPLC calibration curves (against biphenyl as an internal standard) were determined for N-boc-nortropinone, 2-iodopyrimidine and EU1G, and are presented as FIGS. 1, 2, and 3, respectively. An example HPLC calibration chromatogram showing all three reaction components and the internal reference standard (biphenyl), is depicted in FIG. 4. For HPLC analysis, 100 μL of reaction solution was diluted with a mixture of acetonitrile and water (about 1 mL). At least three samples (duration of sampling ~3 residence times per sample) were taken sequentially for each experiment, once steady state had been reached (at least 5 residence times after injection of the reagents). The results from the three samples were averaged for each experiment to determine the amounts of EU1F, EU1G and N-boc-nortropinone.

### Example 1 Flow Setup A

An optimisation experiment was performed with Setup A (FIG. 5).

TABLE 1
Results obtained with Set-up A.

| | Flow Rate (mL/min) | | | | | HPLC Yield (%)^{a} | | |
|---|---|---|---|---|---|---|---|---|
| | | | N-boc- | Residence | | | N-boc- | |
| | | | nortro- | Time (s) | | | Nortro- | |
| Entry | EU1F | HexLi | pinone | RT1 | RT2 | EU1F | pinone | EU1G |
| 1 | 1.30 | 0.169 | 0.975 | 6.2 | 4.7 | 3 | 143 | 9 |
| 2 | 2.50 | 0.325 | 1.88 | 3.2 | 2.5 | 6 | 148 | 8 |
| 3^{b} | 1.30 | 0.169 | 0.975 | 6.2 | 4.7 | <1 | 139 | 17 |

^{a}HPLC yield determined using calibration vs biphenyl as internal standard, percentages are relative to the initial amount of EU1F that was added to the reaction.
^{b}2-MeTHF as reaction solvent instead of THF.
RT1 = residence time between the flow junction with HexLi and the flow junction with N-boc-nortropinone (V1, 152 μL volume);
RT2 = residence time between the flow junction with N-boc-nortropinone and the flow junction with citric acid (V2, 192 μL volume).

Table 1 shows the results from the experiments using setup A. Initial conditions (Entry 1) showed low yield of EU1G, high conversion of EU1F, but low conversion of N-boc-nortropinone. These results imply that EU1F reacts quickly with HexLi, but the lithiated intermediate then decomposes or is consumed in side reactions during RT1, before N-boc-nortropinone is added.

Increasing flow rates, thus shortening the residence times for both steps, and improving the mass transfer at the mixing points, did not lead to improvement of the yield of EU1G (Entry 2).

To assess the influence of reaction solvent, the reaction was performed in 2-MeTHF as solvent, instead of THF (Entry 3). Under these conditions, slightly higher yield of EU1G (17%) was observed, but the yield was still much lower than under batch conditions.

For all conditions (Entries 1-3), solid formation was observed visually in the mixing structures after the mixing point of EU1F and HexLi. The switch of solvents to 2-MeTHF (Entry 3) resulted in faster solid formation, which led to clogging of the reactor after 5 minutes of operation.

Due to the observed solid formation and low yields that were achieved under the trialed conditions, the decision was made to switch to an alternative set-up where EU1F and N-boc-nortropinone were introduced in one feed, before being mixed with HexLi (Setup B).

### Example 2 Flow Setup B

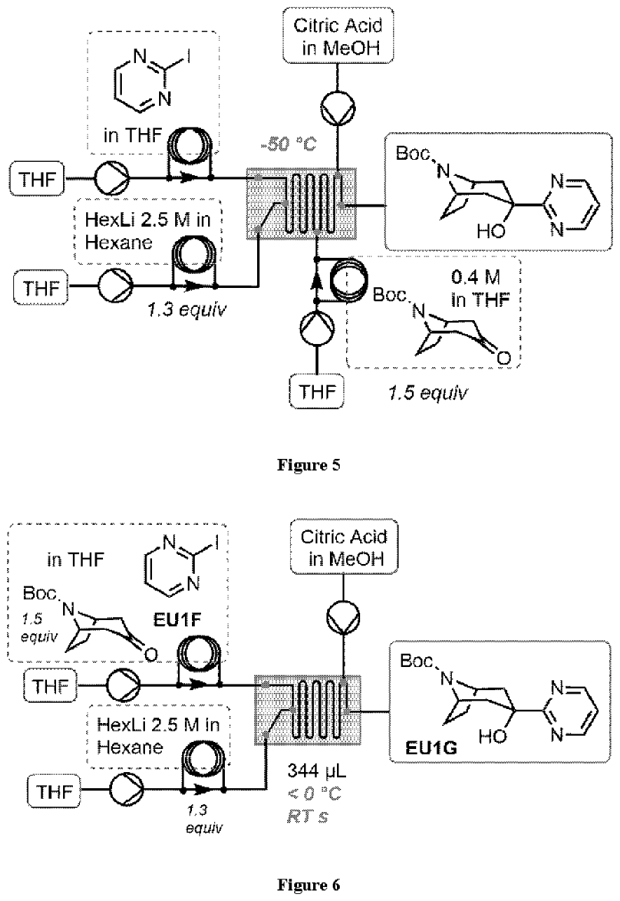

An optimisation experiment was Performed with setup B (FIG. 6)

TABLE 2
Results obtained with setup B.

| | | Flow rate | | | HPLC Yield (%)^{a} | | |
|---|---|---|---|---|---|---|---|
| | | (mL/min) | | RT | | N-boc- | |
| Entry | T (° C.) | EU1F | HexLi | (s) | EU1F | nortropinone | EU1G |
| 1 | −50 | 1.30 | 0.169 | 14.1 | 41 | 103 | 27 |
| 2 | −50 | 2.50 | 0.325 | 7.3 | 12 | 74 | 50 |
| 3 | −30 | 1.30 | 0.169 | 14.1 | 36 | 96 | 26 |
| 4 | 0 | 1.30 | 0.169 | 14.1 | 39 | 103 | 19 |

^{a}HPLC yield determined using calibration vs biphenyl as internal standard, percentages are relative to the initial amount of EU1F that was added to the reaction.
RT = residence time between the flow junction with HexLi and the flow junction with citric acid.

Table 2 shows the results of the reactions performed using setup B. For all reactions (Entries 1-4), more stable reactions regarding possible reactor blockages were observed and no pressure increase or solid formation was observed. Entry 1 shows 27% yield of EU1G and incomplete conversion of EU1F (41% left). Increasing the flow rates (thus decreasing the residence time) led to a much higher yield of 50% EU1G while also improving the conversion of EU1F (Entry 2). This behaviour shows that the incomplete conversion of EU1F is not caused by the residence time being too low, but because HexLi is consumed in side reactions caused by inefficient mixing. Since the mixing is improved by the higher flow rates this also leads to improved conversion and yield.

Entries 3 and 4 were performed at temperatures higher than −50° C. These conditions led to a lower yield when compared to Entry 1, thus showing the temperature dependence of the reaction. This temperature dependence is because side reactions are sped up by the higher temperature, which in turn decreases the selectivity toward product EU1G.

### Example 3—Flow Setup C

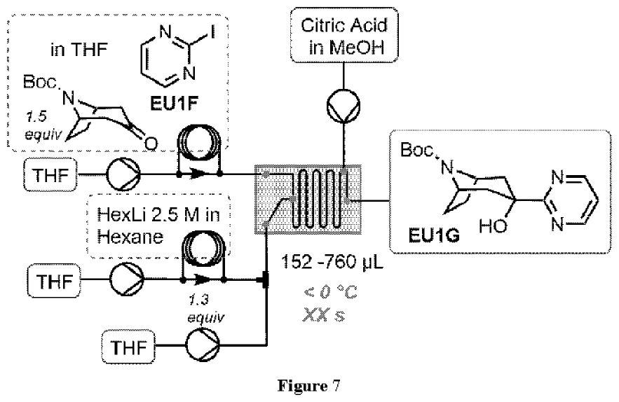

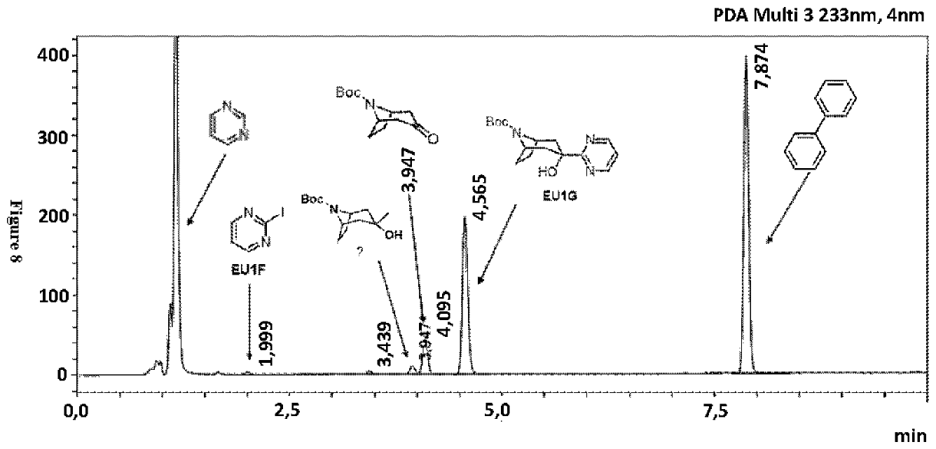

An optimisation experiment was performed with setup C (FIG. 7).

TABLE 3
Results obtained with setup C.

| | | Flow rate | | | | HPLC Yield (%)^{b} | | |
|---|---|---|---|---|---|---|---|---|
| | T | (mL/min) | | V | RT | | N-boc- | |
| Entryª | (° C.) | EU1F | HexLi | (μL) | (s) | EU1F | Nortropinone | EU1G |
| 1 | −50 | 2.50 | 1.63 | 344 | 5.0 | <1 | 71 | 58 |
| 2 | −50 | 2.50 | 1.63 | 152 | 2.2 | 1 | 62 | 58 |
| 3 | −50 | 2.50 | 1.63 | 760 | 11 | 5 | 64 | 57 |
| 4 | −20 | 2.50 | 1.63 | 344 | 5.0 | 2 | 48 | 16 |
| 5 | −50 | 5.00 | 3.25 | 344 | 2.5 | <1 | 71 | 60 |
| 6 | −40 | 5.00 | 3.25 | 344 | 2.5 | 16 | 78 | 48 |
| 7 | −20 | 5.00 | 3.25 | 344 | 2.5 | 16 | 72 | 44 |

ªReaction conditions: 1.5 equiv N-boc-nortropinone, 0.25 M EU1F concentration in feed solution,
^{b}HPLC yield determined using calibration vs biphenyl as internal standard, percentages are relative to the initial amount of EU1F that was added to the reaction.
RT = residence time between the flow junction with HexLi and the flow junction with citric acid.

Table 3 shows the results of the initial experiments performed using setup C. When using similar conditions to the previous best results (Entry 2 of Table 2) in Entry 1, but with HexLi being diluted to 1.5 M with THF, the yield was improved to 58% EU1G. When the residence time was varied by moving the quench introduction port, while keeping the flow rates the same (Entries 2 and 3), only minor differences in yield were observed (58 and 57% EU1G). Entry 4 shows that under the same conditions but at −20° C., a large drop in yield to 16% EU1G was observed. Doubling the flow rates to 5.00 mL/min EU1F (shortening the residence time to 2.5 s) was feasible, yielding 60% EU1G (Entry 5). When increasing temperature to −40° C. (Entry 6) and −20° C. (Entry 7) at the same flow rates, a drop in yield to 48% and 44%, respectively, was observed. Notably, at higher flow rates much better results were achieved than at lower flow rates (entry 4 vs entry 7).

### Example 4—Screen of Organolithium Reagents

An optimisation experiment screening different organolithium reagents (RLi) was performed with setup C.

TABLE 4
Results of the screen of different organolithium reagents.

| | | Flow rate | | | HPLC Yield (%)^{b} | | |
|---|---|---|---|---|---|---|---|
| | Reagent | (mL/min) | | RT | | N-boc- | |
| Entryª | (RLi) | EU1F | RLi | (s) | EU1F | Nortropinone | EU1G |
| 1 | HexLi | 2.50 | 1.63 | 5.0 | <1 | 71 | 58 |
| 2 | n-BuLi | 2.50 | 1.63 | 5.0 | 3 | 71 | 59 |
| 3 | PhLi | 2.50 | 1.63 | 5.0 | 19 | 46 | 48 |
| 4 | MeLi | 2.50 | 1.63 | 5.0 | <1 | 58 | 69 |
| 5 | MeLi•LiBr | 2.50 | 1.63 | 5.0 | 2 | 57 | 71 |
| 6 | MeLi•LiBr | 5.00 | 3.25 | 2.5 | 4 | 55 | 72 |

^{a}Reaction conditions: −50° C., 1.3 equiv RLi reagent, 1.5 equiv N-boc-nortropinone, reactor volume = 344 μL, 0.25 M EU1F concentration in feed solution,
^{b}HPLC yield determined using calibration vs biphenyl as internal standard, percentages are relative to the initial amount of EU1F that was added to the reaction.
RT = residence time between the flow junction with HexLi and the flow junction with citric acid.

Table 4 shows the results of a screen of different organolithium reagents under otherwise constant conditions. Hexyllithium (Entry 1, HexLi) and n-butyllithium (Entry 2, n-BuLi) show similar results (58% and 59% yield). Phenyllithium (Entry 3, PhLi) leads to only 81% conversion of EU1F and a drop in yield to 48%. Methyllithium (Entry 4, MeLi) and methyllithium lithium bromide complex (Entry 5, MeLi·LiBr) showed a significantly improved yield (69% and 71% EU1G, respectively). Using MeLi·LiBr, doubling the flow rates to improve mixing showed a minor improvement to 72% EU1G, while proving that a residence time of 2.5 s is enough to complete the reaction.

### Example 5—Screen of Organolithium Reagent Equivalents

An optimisation experiment screening different equivalents of organolithium reagent (RLi) was performed with setup C

TABLE 5
Results of the screen of different organolithium reagent equivalents.

| | | Flow rate | | | HPLC Yield (%)^{b} | | |
|---|---|---|---|---|---|---|---|
| | Reagent | (mL/min) | | RT | | N-boc- | |
| Entryª | (equiv.) | EU1F | RLi | (s) | EU1F | Nortropinone | EU1G |
| 1 | HexLi | 2.50 | 1.38 | 5.3 | 15 | 79 | 54 |
| | (1.1) | | | | | | |
| 2 | HexLi | 2.50 | 1.63 | 5.0 | <1 | 71 | 58 |
| | (1.3) | | | | | | |
| 3 | HexLi | 2.50 | 2.00 | 4.6 | <1 | 57 | 56 |
| | (1.6) | | | | | | |
| 4 | MeLi•LiBr | 2.50 | 1.38 | 5.3 | 10 | 77 | 63 |
| | (1.1) | | | | | | |
| 5 | MeLi•LiBr | 2.50 | 1.63 | 5.0 | 2 | 55 | 71 |
| | (1.3) | | | | | | |
| 6 | MeLi•LiBr | 2.50 | 2.00 | 4.6 | <1 | 36 | 75 |
| | (1.6) | | | | | | |

^{a}Reaction conditions: −50° C.,1.5 equiv N-boc-nortropinone, 344 μL reactor volume, 0.25 M EU1F concentration in feed solution,
^{b}HPLC yield determined using calibration vs biphenyl as internal standard, percentages are relative to the initial amount of EU1F that was added to the reaction.
RT = residence time between the flow junction with HexLi and the flow junction with citric acid.

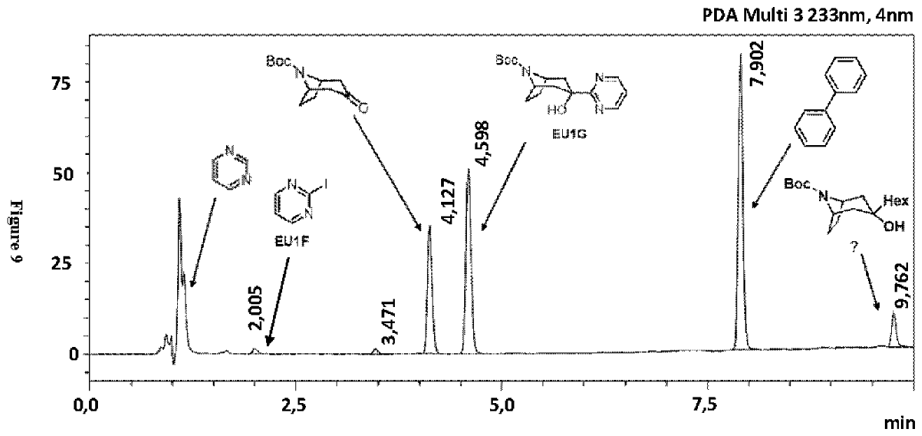

Table 5 shows the results of a screen of organolithium reagent equivalents. Both HexLi and MeLi·LiBr were trialed at 1.1 and 1.6 equiv and compared to the previously used 1.3 equiv. The experiments for HexLi showed no improvement when increasing or decreasing the equivalents (Entries 1-3). For MeLi·LiBr (Entries 4-6), a drop in yield to 63% was seen at 1.1 equiv and an increase to 75% HPLC yield when using 1.6 equiv. FIG. 8 depicts a representative chromatogram of a reaction mixture with MeLi·LiBr. FIG. 9 depicts a representative chromatogram of a reaction mixture with HexLi.

### Example 6—Screen of Temperature and Concentration

An optimisation experiment screening different temperatures and concentration of EU1F was performed with setup C.

TABLE 6
Results of the Screen of temperature and concentration.

| | | | Flow rate | | | HPLC Yield (%)^{c} | | |
|---|---|---|---|---|---|---|---|---|
| | Conc. | T | (mL/min) | | RT | | N-boc- | |
| Entryª | (M)^{b} | (° C.) | EU1F | MeLi•LiBr | (s) | EU1F | Nortropinone | EU1G |
| 1 | 0.10 | −50 | 2.50 | 0.80 | 6.3 | 2 | 51 | 67 |
| 2 | 0.25 | −50 | 2.50 | 2.00 | 4.6 | <1 | 36 | 75 |
| 3 | 0.40 | −50 | 2.50 | 3.20 | 3.6 | <1 | 30 | 75 |
| 4 | 0.25 | −50 | 2.50 | 2.00 | 4.6 | 1 | 31 | 72 |
| 5 | 0.25 | −50 | 2.50 | 2.00 | 4.6 | 1 | 29 | 64 |
| 6 | 0.25 | −50 | 5.00 | 4.00 | 2.3 | <1 | 31 | 68 |
| 7 | 0.25 | −50 | 5.00 | 4.00 | 2.3 | <1 | 29 | 62 |

^{a}Reaction conditions: 1.5 equiv N-boc-nortropinone, 1.6 equiv MeLi•LiBr, 344 μL reactor volume,
^{b}Concentration of EU1F in feed solution,
^{c}HPLC calibrated yield determined usingcalibration vs biphenyl as internal standard, percentages are relative to the initial amount of EU1F that was added to the reaction.
RT = residence time between the flow junction with HexLi and the flow junction with citric acid.

In Table 6, the results of further optimization experiments of temperature and concentration are shown. Lowering the concentration of EU1F in the feed to 0.1 M leads to a drop in yield to 67%. Increasing the concentration to 0.4 M is possible without a drop in yield.

At 0.25 M concentration, increasing the temperature to −40° C. (Entry 4) was possible with a minor drop in yield (72%). Further increasing the temperature to −20° C. (Entry 5) leads to a drop in yield to 64%. Doubling of the flow rates was expected to improve the performance at higher temperatures by improving the mixing. The results in entries 6 and 7 show no improvement of yield at −40 or −20° C. ending up with slightly lower yields of EU1G (68% and 62%, respectively).

### Example 7—Screen of N-Boc-Nortropinone Equivalents

An optimisation experiment screening different equivalents of N-boc-nortropinone was performed with setup C.

TABLE 7
Results of the screen of N-boc-nortropinone equivalents.

| | Equivalents | | Flow rate | | | HPLC Yield (%)^{b} | | |
|---|---|---|---|---|---|---|---|---|
| | N-boc- | | (mL/min) | | RT | | N-boc- | |
| Entryª | nortropinone | MeLi•LiBr | EU1F | MeLi•LiBr | (s) | EU1F | nortropinone | EU1G |
| 1 | 0.9 | 1.1 | 2.50 | 1.38 | 5.3 | 4 | 15 | 61 |
| 2 | 0.9 | 1.3 | 2.50 | 1.63 | 5.0 | <1 | 15 | 44 |
| 3 | 0.9 | 1.6 | 2.50 | 2.00 | 4.6 | <1 | 14 | 36 |
| 4 | 0.9 | 2.0 | 2.50 | 2.50 | 4.1 | <1 | 13 | 28 |
| 5 | 1.2 | 1.1 | 2.50 | 1.38 | 5.3 | 4 | 36 | 71 |
| 6 | 1.2 | 1.3 | 2.50 | 1.63 | 5.0 | <1 | 22 | 71 |
| 7 | 1.2 | 1.6 | 2.50 | 2.00 | 4.6 | <1 | 22 | 58 |
| 8 | 1.2 | 2.0 | 2.50 | 2.50 | 4.1 | <1 | 21 | 45 |
| 9 | 1.5 | 1.1 | 2.50 | 1.38 | 5.3 | 10 | 77 | 63 |
| 10 | 1.5 | 1.3 | 2.50 | 1.63 | 5.0 | 2 | 55 | 71 |
| 11 | 1.5 | 1.6 | 2.50 | 2.00 | 4.6 | <1 | 36 | 75 |
| 12 | 1.5 | 2.0 | 2.50 | 2.50 | 4.1 | <1 | 26 | 56 |
| 13 | 1.8 | 1.1 | 2.50 | 1.38 | 5.3 | 7 | 100 | 66 |
| 14 | 1.8 | 1.3 | 2.50 | 1.63 | 5.0 | 1 | 83 | 71 |
| 15 | 1.8 | 1.6 | 2.50 | 2.00 | 4.6 | <1 | 60 | 71 |
| 16 | 1.8 | 2.0 | 2.50 | 2.50 | 4.1 | <1 | 32 | 68 |

ªReaction conditions: −50° C., reactor volume = 344 μL, 0.25 M EU1F in the feed solution,
^{b}HPLC yield determined using calibration vs biphenyl as internal standard, percentages are relative to the initial amount of EU1F that was added to the reaction.
RT = residence time between the flow junction with HexLi and the flow junction with citric acid.

Table 7 shows the results of a screen of N-boc-nortropinone equivalents (0.9-1.8 equiv). For each level of N-boc-nortropinone equiv, four different equivalents of MeLi·LiBr were screened (16 experiments in total).

Entries 1-4 show the results with 0.9 equiv of N-boc-nortropinone. A clear trend is seen that the yield of EU1G decreases with higher excess of MeLi·LiBr reagent. In general, a lower yield is observed with a maximum of 61% (Entry 1).

Entries 5-8 show the results with 1.2 equiv N-boc-nortropinone. A similar trend was observed, showing the highest yield (71%) with 1.1 and 1.3 equiv of MeLi·LiBr. The yield then decreases with higher excess of organolithium reagent.

Entries 9-12 show the results with 1.5 equiv N-boc-nortropinone. Here, the previously best conditions of 1.6 equiv MeLi·LiBr give the highest yield (75%). Both at higher and lower equivalents the yield decreases.

Entries 13-16 show the results for 1.8 equivalents of N-boc-nortropinone. The highest yield is achieved with 1.3 and 1.6 equivalents of MeLi·LiBr (71%) while higher and lower equivalents show slightly decreased yield.

After these final optimisation experiments two sets of conditions were selected as preferable for further experimentation:
(1) 1.5 equiv N-boc-nortropinone and 1.6 equiv MeLi·LiBr (75% yield); and
(2) 1.2 equiv N-boc-nortropinone and 1.3 equiv MeLi·LiBr (71% yield of EU1G with significantly lower equivalents—may be preferable).

### Example 8—Further Validation Experiments

To show that the optimised conditions are feasible for larger scale experiments, some validation experiments that focus on high yield and productivity were performed.

TABLE 8
Validation experiments of final conditions at 0.4 M concentration.

| | Equivalents | | Flow rate | | | HPLC Yield (%)^{b} | | |
|---|---|---|---|---|---|---|---|---|
| | N-boc- | | (mL/min) | | RT | | N-boc- | |
| Entryª | nortropinone | MeLi•LiBr | EU1F | MeLi•LiBr | (s) | EU1F | nortropinone | EU1G |
| 1 | 1.2 | 1.2 | 2.50 | 2.40 | 4.2 | 1 | 27 | 69 |
| 2 | 1.2 | 1.3 | 2.50 | 2.60 | 4.0 | <1 | 21 | 68 |
| 3 | 1.5 | 1.5 | 2.50 | 3.00 | 3.8 | <1 | 29 | 70 |
| 4 | 1.5 | 1.6 | 2.50 | 3.20 | 3.6 | <1 | 27 | 66 |

^{a}Reaction conditions: −50° C., reactor volume = 344 μL, 0.4 M EU1F in the feed solution,
^{b}HPLC yield determined using calibration vs biphenyl as internal standard, percentages are relative to the initial amount of EU1F that was added to the reaction.
RT = residence time between the flow junction with HexLi and the flow junction with citric acid.

Table 8 shows the results of these initial validation experiments. To improve the productivity, 0.4 M concentration of EU1F was employed in all reactions. Entry 1 was performed using 1.2 equiv of both N-boc-nortropinone and MeLi·LiBr. The reaction performed slightly worse than previously observed in optimization experiments (entry 1, 69% instead of 71% yield, using similar conditions). An increase to 1.3 equiv of MeLi·LiBr to exactly mimic the conditions from Table 7 did not lead to an increased yield (entry 2, 68%). Entries 3 and 4 show a similar drop in yield (70% and 66% yield) when compared to previous experiments (75%).

To account for the decreased yields that were observed in Table 8, a search for the cause was performed. Notably, the substrate EU1F showed a darker yellow color compared to when it was delivered. A re-measurement of the purity of EU1F using NMR with internal standard showed only 93% purity. This explains the lower yields shown in Table 8—a lower amount of substrate EU1F was present in the reaction from the start, because of the lower purity.

To avoid the introduction of any water to the reaction, EU1F was always dried before use, but the bulk of the material was stored under air. The degradation of EU1F shows that the compound needs to be stored under argon at all times.

TABLE 9
Validation experiments of final conditions at 0.4 M concentration, corrected for purity of EU1F.

| | Equivalents | | Flow rate | | | HPLC Yield (%)b | | |
|---|---|---|---|---|---|---|---|---|
| | N-boc- | | (mL/min) | | RT | | N-boc- | |
| Entryª | nortropinone | MeLi•LiBr | EU1F | MeLi•LiBr | (s) | EU1F | nortropinone | EU1G |
| 1 | 1.27 | 1.27 | 2.50 | 2.40 | 4.2 | 1 | 27 | 73 |
| 2 | 1.27 | 1.38 | 2.50 | 2.60 | 4.0 | <1 | 21 | 72 |
| 3 | 1.59 | 1.59 | 2.50 | 3.00 | 3.8 | <1 | 29 | 74 |
| 4 | 1.59 | 1.70 | 2.50 | 3.20 | 3.6 | <1 | 27 | 70 |

^{a}Reaction conditions: −50° C., reactor volume = 344 μL, 0.4 M EU1F in the feed solution,
^{b}HPLC yield determined using calibration vs biphenyl as internal standard, percentages are relative to the initial amount of EU1F that was added to the reaction, which was corrected for the purity of EU1F.
RT = residence time between the flow junction with HexLi and the flow junction with citric acid.

Table 9 shows the same results as in Table 8, but taking into account the decreased purity of the EU1F. This leads to a slight change in the equivalents and in an improved yield of EU1G when considering that less starting material was present.

Entries 1 and 3 showed the most promising results for longer stability runs with 73 and 74% yield, respectively. The conditions from Entry 1 were selected as most suitable, since the yield is almost the same as in Entry 3, while employing significantly lower equivalents of reagents. To avoid miscalculations of the yield of product, because of decreased purity of starting material, HPLC measurements of the feed solution for all further experiments were performed to determine the amount of EU1F present in the starting solution for every reaction. The calculated yields from this point on are corrected to take this concentration into account.

### Example 9—Demonstration of Stability in Longer Runs and Isolation

Stability of the flow system over a prolonged duration was demonstrated in three stability experiments. All 6-port valves and sample loops were removed from the set-up and all substrate and reagent streams were pumped directly using syringe pumps.

The following three solutions (Feed 1, Feed 2, and Quench Solution) were prepared as follows:
Feed 1: 4.148 g of EU1F (19.2 mmol, considering 95.5% purity), 5.437 g N-boc-nortropinone (24.1 mmol, 1.25 equiv) and 0.633 g biphenyl (4.1 mmol, 0.21 equiv) were placed in a 50 mL volumetric flask. The flask was placed under argon atmosphere and brought to volume with dry THF. Feed 2: 17.33 mL of MeLi·LiBr (1.5 M in Et₂O) were diluted with THF in a 50 mL volumetric flask under argon (0.52 M final concentration). Quench Solution: 26.27 g of citric acid was dissolved in 500 mL methanol to reach a final concentration of 0.25 M.

Feed 1 and 2 were kept under argon at all times, using balloons. When not in use, all the pumps and tubing were stored under iPrOH (for compatibility reasons). Prior to starting experiments, the syringe pumps, reactor and all tubing were flushed with the solvent (dry THF) for at least 10 minutes at the respective maximum flow rates for each pump.

A first stability experiment was performed on 20 mmol scale (corresponding to −20 min operation at 0.4 M concentration and 2.5 mL/min substrate flow rate). The experiment was performed using 1.25 equiv MeLi·LiBr and 1.25 equiv N-boc-nortropinone (already adjusted for EU1F purity). The first stability run resulted in 60% yield of EU1G over the whole operation time of 19 minutes and only 72% conversion of EU1F. This decrease compared to previous experiments can be explained by MeLi·LiBr not being pumped correctly with the modified set-up. Pumping the solution of MeLi·LiBr in Et₂O directly using a syringe pump resulted in cavitation of solvent caused by the high vapor pressure of diethyl ether. This provided an incorrect volume flow rate in the experiment. Decomposition of the organolithium species within the syringe is also a possible reason for the decrease in yield.

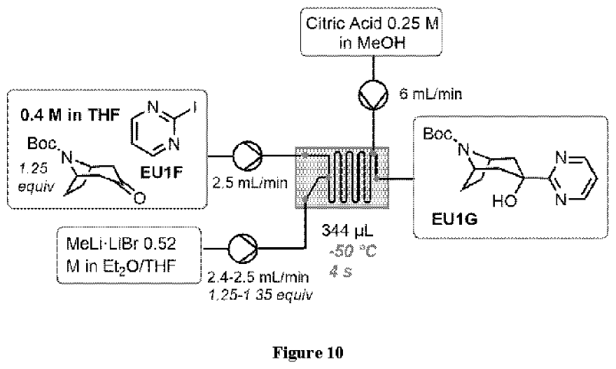

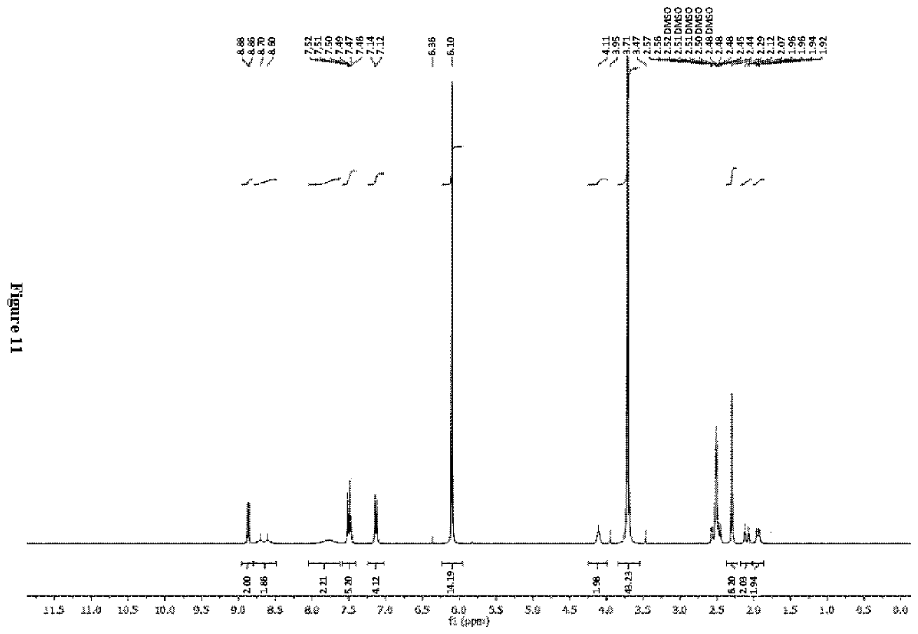

To address the problem of Et₂O cavitating when being pumped, a workaround was developed by adjusting the set-up. FIG. 10 shows the flow set-up that was used to perform further long run experiments (setup D). The dilution stream introduced into setup C was removed, when switching to setup D, opting instead for a lower final concentration of Feed 2 (MeLi·LiBr). Specifically, MeLi·LiBr was prediluted with THF to a final concentration of 0.5 M using THF to decrease the vapour pressure of the mixture and avoid cavitation. The additional THF pump was taken out of the setup, as it was no longer needed to dilute MeLi·LiBr.

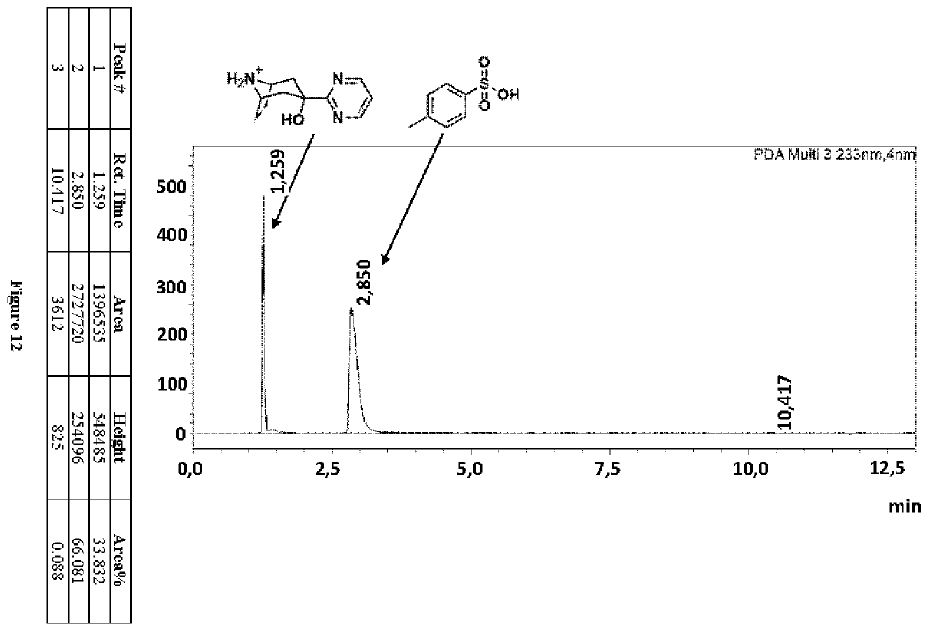

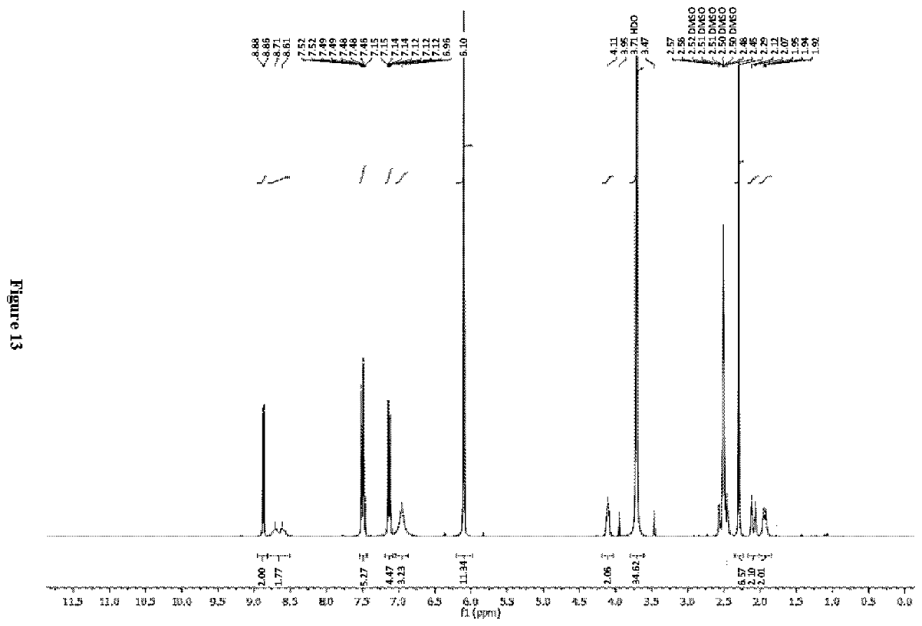

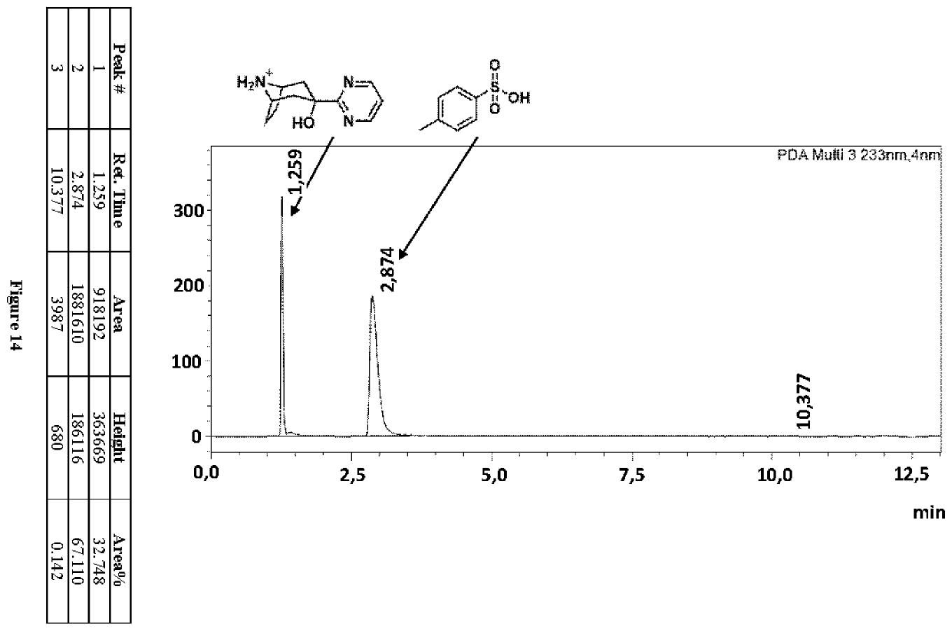

In this first isolation experiment, MeLi·LiBr was fed at a rate of 2.4 mL/min, with all other variables kept unchanged from the first run. After 8 min of steady state collection (71% HPLC yield), the product was isolated as the boc-deprotected pTSA salt. Briefly, methanol was evaporated from the solution using a rotary evaporator, and the solution was diluted with ethyl acetate then washed twice with water/brine. The aqueous phase was back extracted once using ethyl acetate. The combined organic phases were dried over Na₂SO₄. The solvent was then evaporated and replaced with 2-MeTHF. After heating to 50° C., 3.5 equivalents of pTSA in 2-MeTHF was added. After approximately 15 min, a white precipitate formed. After 2.5 h, the product was isolated by filtration and washed with MeTHF, then 40-60 petroleum ether. 2.6 g (63%) of the pTSA salt was isolated, with an NMR purity of 95%. This isolated yield corresponds to a productivity of 19.5 g/h and a space-time yield of 56.7 kg/L/h, based on the flow reaction. A ¹H NMR spectrum and HPLC trace of the isolated boc-deprotected pTSA product are presented as FIG. 11 and FIG. 12 respectively. A second isolation experiment was performed with 1.35 equivalents of MeLi·LiBr (a flow rate of 2.5 mL/min) with all other variables kept the same. 20 minutes of steady state collection resulted in a 72% HPLC yield prior to boc deprotection, and 6.0 g (64%) isolated yield of the boc-deprotected pTSA salt, with an NMR purity of 91%. A ¹H NMR spectrum and HPLC trace of the isolated boc-deprotected pTSA product are presented as FIG. 13 and FIG. 14 respectively.

1. A continuous flow process for preparing an aza-bicyclic compound of Formula 1, or salt thereof: 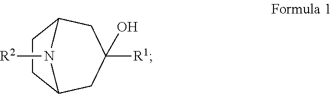
comprising an organolithium reaction of a nortropinone compound of Formula 2: 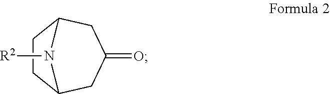
with a halogenated compound of Formula 3: 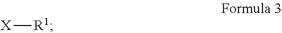
in the presence of an organolithium reagent;
wherein
R¹ is a monocyclic or bicyclic heteroaryl group each unsubstituted or substituted with one or more substituents selected from the group consisting of halogen, —C₁₋₆alkyl, —O—C₁₋₆alkyl, —C₁₋₆haloalkyl, and —O—C₁₋₆haloalkyl;
R² is an amine protecting group; and
X is a halogen.

2. The continuous flow process of claim 1, wherein the continuous flow process comprises the nortropinone compound of Formula 2, the halogenated compound of Formula 3, and the organolithium reagent, in one or more continuous flow streams, in a continuous flow reactor configured at a temperature effective for synthesising the aza-bicylic compound of Formula 1, or salt thereof.

3. The continuous flow process of claim 1 or claim 2, wherein the nortropinone compound of Formula 2 and the halogenated compound of Formula 3 are initially combined.

4. The continuous flow process of any one of claims 1 to 3, wherein a combination of the nortropinone compound of Formula 2 and the halogenated compound of Formula 3, is subsequently combined with the organolithium reagent.

5. The continuous flow process of any one of claims 1 to 4, wherein the process comprises:
combining a continuous flow stream comprising the compound of Formula 2 and the compound of Formula 3, with a continuous flow stream comprising the organolithium reagent, to obtain a continuous flow stream comprising an aza-bicyclic compound of Formula 1, or salt thereof.

6. The continuous flow process of any one of claims 1 to 5, wherein the process comprises:
combining a continuous flow stream comprising the compound of Formula 2 with a continuous flow stream comprising the compound of Formula 3, to obtain a continuous flow stream comprising the compound of Formula 2 and the compound of Formula 3; and
combining the continuous flow stream comprising the compound of Formula 2 and the compound of Formula 3, with a continuous flow stream comprising the organolithium reagent, to obtain a continuous flow stream comprising an aza-bicyclic compound of Formula 1, or salt thereof.

7. The continuous flow process of claim 1 or claim 2, wherein the process comprises:
combining a continuous flow stream comprising a compound of Formula 3 with a continuous flow stream comprising an organolithium reagent, to obtain a continuous flow stream comprising an organolithium reaction intermediate; and
combining the continuous flow stream comprising the organolithium reaction intermediate with a continuous flow stream comprising a compound of Formula 2, to obtain a continuous flow stream comprising an aza-bicyclic compound of Formula 1, or salt thereof.

8. The continuous flow process of any one of claims 1 to 7, wherein the continuous flow process further comprises:
combining the aza-bicyclic compound of Formula 1, or salt thereof, with a quenching solution.

9. The continuous flow process of claim 8, wherein the quenching solution is selected from the group consisting of: a polar protic solvent, a non-polar protic solvent, an aqueous solvent, an alcohol, and an acid, or a combination thereof.

10. The continuous flow process of claim 8 or claim 9, wherein the process comprises:
combining a continuous flow stream comprising the aza-bicyclic compond of Formula 1, or salt thereof, with a continuous flow stream comprising the quenching solution, to obtain a continuous flow stream comprising the aza-bicyclic compound of Formula 1.

11. The continuous flow process of any one of claims 1 to 10, wherein the continuous flow process further comprises:
combining a continuous flow stream comprising a solvent with a continuous flow stream comprising the organolithium reagent, to controllably dilute the concentration of the organolithium reagent.

12. The continuous flow process of any one of claims 1 to 11, wherein the continuous flow process further comprises:
preparing a salt of an amine bicyclic compound of Formula 4: 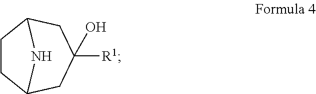
wherein the process comprises reacting a compound of Formula 1 with an acid to remove the amine protecting group, to obtain a salt of the compound of Formula 4.

13. The continuous flow process of any one of claims 1 to 12, wherein the continuous flow process further comprises:
preparing a heterocyclic methanone compound of Formula 5: 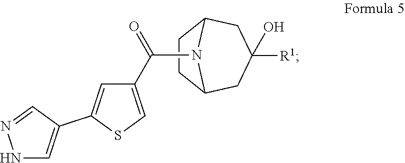
wherein the process comprises reacting a carboxylic acid compound of Formula 6, or a salt thereof: 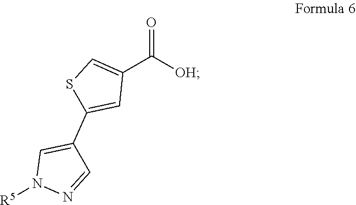
with the amine bicyclic compound of Formula 4, or salt thereof, in the presence of at least one coupling reagent, to obtain the heterocyclic methanone compound of Formula 5.

14. The continuous flow process of any one of claims 1 to 13, wherein the organolithium reagent is selected from any compound containing a carbon-lithium bond or a carbon-magnesium-lithium bond.

15. The continuous flow process of any one of claims 1 to 14, wherein the organolithium reagent is selected from the group consisting of: alkyl lithium, alkenyl lithium, alkynyl lithium, aralkyl lithium, aryl lithium, heteroaryl lithium, alkyllithium magnesium complexes, and any lithium halide complexes thereof.

16. The continuous flow process of any one of claims 1 to 15, wherein the organolithium reagent is selected from the group consisting of: methyllithium, ethyllithium, propyllithium, butyllithium, pentyllithium, hexyllithium, methoxymethyllithium, ethoxymethyllithium, vinyllithium, allyllithium, propenyllithium, butenyllithium, ethynyllithium, butynyllithium, pentynyllithium, hexynyllithium, benzyllithium, phenylethyllithium, phenyllithium, naphthyllithium, 2-thienyllithium, 4-pyridyllithium, 2-quinolyllithium, tri(n-butyl)magnesiumlithium and trimethylmagnesiumlithium, (trimethylsilyl)methyllithium, lithium(trimethylsilyl) acetylide, lithium acetylide, lithium phenylacetylide, cyclopentadienyllithium, lithium pentamethylcyclopentadienide, lithium methide, and any lithium halide complexes thereof.

17. The continuous flow process of any one of claims 1 to 16, wherein the organolithium reagent is selected from the group consisting of: methyllithium or any lithium halide complexes thereof.

18. The continuous flow process of any one of claims 1 to 17, wherein the amount of the organolithium reagent relative to the amount of the halogenated compound of Formula 3 is provided in a Molar ratio of between about 0.1 to about 10.

19. The continuous flow process of any one of claims 1 to 18, wherein the concentration of the organolithium reagent is at least about 0.1 mol/L.

20. The continuous flow process of any one of claims 1 to 19, wherein the one or more continuous flow streams each has a flow rate of at least about 0.5 mL/min.

21. The continuous flow process of any one of claims 1 to 20, wherein the amount of the nortropinone compound of Formula 2 relative to the amount of the halogenated compound of Formula 3, is provided in a Molar ratio of between about 0.1 to about 10.

22. The continuous flow process of any one of claims 1 to 21, wherein the residence time between obtaining the continuous flow stream comprising the organolithium reaction intermediate and obtaining a continuous flow stream comprising the aza-bicyclic compound of Formula 1, or salt thereof, is at least 0.01 seconds.

23. The continuous flow process of any one of claims 1 to 22, wherein the process is performed at a temperature of less than 20° C., less than 10° C., less than 0° C., less than −10° C., less than −20° C., less than −30° C., less than −40° C., less than −50° C., less than −60° C., less than −70° C., or less than −80° C.

24. The continuous flow process of any one of claims 1 to 23, wherein the two or more continuous flow streams are provided as solutions comprising at least one solvent independently selected from the group consisting of: tetrahydrofuran, methyl tetrahydrofuran, diethyl ether, methyl tert-butyl ether, cyclopentyl methyl ether, toluene, and combinations thereof.

25. The continuous flow process of any one of claims 1 to 24, wherein the process provides the aza-bicylic compound of Formula 1 in a yield of at least 20%, at least 30%, at least 40%, at least 50%, at least 60%, at least 70%, at least 80%, or at least 90%, as determined from a compound of Formula 2 or a compound of Formula 3 starting material.

26. The continuous flow process of any one of claims 1 to 25, wherein the process provides a conversion of the nortropinone compound of Formula 2, or of the halogenated compound of Formula 3, to an aza-bicyclic compound of Formula 1, or salt thereof, of at least 20%, at least 30%, at least 40%, at least 50%, at least 60%, at least 70%, at least 80%, or at least 90%, as measured by HPLC.

27. The continuous flow process of any one of claims 1 to 26, wherein the process provides the aza-bicylic compound of Formula 1 in at least 70%, at least 80%, at least 90%, or at least 95% purity, as determined by HPLC.

28. The continuous flow process of any one of claims 1 to 27, wherein the process takes place in a reactor of a type selected from the group consisting of: continuous flow reactor; microreactor; continuous tubular reactor; laminar flow reactor; flow reactor; static mixing elements; continuous stirred tank reactor; and semibatch reactor.
